(19) United States
(12) Patent Application Publication
(10) Pub. No.: US 2025/0281755 A1
(43) Pub. Date: Sep. 11, 2025

(54) METHOD AND APPARATUS FOR CONDUCTION SYSTEM PACING CAPTURE CLASSIFICATION

(71) Applicant: Medtronic, Inc., Minneapolis, MN (US)

(72) Inventors: Keara A. BERLIN, St. Paul, MN (US); Jian CAO, Shoreview, MN (US); Tarek D. HADDAD, Minneapolis, MN (US); Kelvin MEI, Minneapolis, MN (US)

(21) Appl. No.: 18/861,874

(22) PCT Filed: Apr. 25, 2023
(86) PCT No.: PCT/IB2023/054234

§ 371 Date: Oct. 30, 2024

Related U.S. Application Data
(60) Provisional application No. 63/337,769, filed on May 3, 2022.

(51) Int. Cl. A61N 1/365; A61N 1/372

(52) U.S. Cl. CPC A61N 1/365; A61N 1/37247

(57) ABSTRACT

A medical device system includes a memory configured to store a cardiac signal sensed following delivery of a ventricular conduction system pacing pulse. The medical device system includes processing circuitry configured to determine a capture type classification of the ventricular conduction system pacing pulse and generate an output based on the capture type classification. The medical device system may include a user interface configured to present a representation of the capture type classification associated with the delivered ventricular conduction system pacing pulse.

### TECHNICAL FIELD

This disclosure relates to a method and apparatus for classifying the type of capture of cardiac conduction system pacing pulses.

### BACKGROUND

During normal sinus rhythm (NSR), the heartbeat is regulated by electrical signals produced by the sino-atrial (SA) node located in the right atrial wall. Each intrinsic atrial depolarization signal produced by the SA node spreads across the atria, causing the depolarization and contraction of the atria, and arrives at the atrioventricular (AV) node. The AV node responds by propagating a ventricular depolarization signal through the Bundle of His (or “His bundle”) of the ventricular septum and thereafter to the Purkinje branches and the Purkinje muscle fibers of the right and left ventricles. This native conduction system including the His bundle, right and left branches (sometimes referred to as the right and left bundle branches) and the Purkinje fibers may be referred to as the “His-Purkinje conduction system” or “His-Purkinje system.”

Patients with a conduction system abnormality, e.g., poor AV node conduction, poor SA node function, or other conduction abnormalities, may receive a pacemaker to restore a more normal heart rhythm and AV synchrony. Ventricular pacing may be performed to maintain the ventricular rate in a patient having conduction abnormalities. A single chamber ventricular pacemaker may be coupled to a transvenous ventricular lead carrying electrodes placed in the right ventricle, e.g., in the right ventricular apex. The pacemaker itself is generally implanted in a subcutaneous pocket with the transvenous ventricular lead tunneled to the subcutaneous pocket. Intracardiac pacemakers have been introduced or proposed for implantation entirely within a patient's heart, eliminating the need for transvenous leads. An intracardiac pacemaker may provide sensing and pacing from within a chamber of the patient's heart, e.g., from within the right ventricle in a patient having AV conduction block or other conduction abnormalities to provide ventricular rate support.

Dual chamber pacemakers are available which include a transvenous atrial lead carrying electrodes which are placed in the right atrium and a transvenous ventricular lead carrying electrodes that are advanced through the right atrium into the right ventricle. A dual chamber pacemaker senses atrial electrical signals and ventricular electrical signals and can provide both atrial pacing and ventricular pacing as needed to promote a normal atrial and ventricular rhythm and promote AV synchrony when SA and/or AV node or other conduction abnormalities are present.

Cardiac pacing of the His-Purkinje system has been proposed to provide ventricular pacing along the heart's native conduction system. Chronic ventricular pacing via electrodes at or near the right ventricular apex may be associated with increased risk of atrial fibrillation or heart failure. Alternative pacing sites along the His-Purkinje system have been investigated or proposed, such as pacing at or near the His bundle or in an area proximate the left and/or right bundle branches. Pacing the ventricles via the His-Purkinje system of the heart allows recruitment along the heart's natural conduction system and is hypothesized to promote more physiologically normal cardiac activation than other pacing sites, such as the ventricular apex.

### SUMMARY

The techniques of this disclosure generally relate to a medical device system configured to classify the capture type of cardiac pacing pulses delivered to the His-Purkinje conduction system of a patient's heart. The type of capture of a delivered His-Purkinje conduction system pacing pulse, also referred to herein as a “ventricular conduction system” (VCS) pacing pulse, is classified by processing circuitry of a medical device system as one of multiple capture types. Among the capture type classification are, for example, selective His-Purkinje system capture without ventricular myocardial capture, non-selective His-Purkinje system capture with ventricular myocardial capture, ventricular myocardial capture without His-Purkinje system capture, or loss of capture. The processing circuitry of the medical device system may receive cardiac signals obtained during His-Purkinje system pacing. The processing circuitry may extract data from the received cardiac signals for applying an artificial intelligence or machine learning model to the extracted data. The model may be trained using cardiac signal datasets to classify a cardiac signal, e.g., a post-pace cardiac signal waveform, and associated VCS pacing pulse as one of multiple capture types in some examples. The processing circuitry may generate pacing capture information based on capture classifications determined based on the machine learning model for visual representation to a user in a user interface. Additionally or alternatively, the processing circuitry may generate pacing capture information based on the capture classifications for use in programming operating parameters into and controlling VCS pacing by a medical device. Such pacing capture information may include but is not limited to capture thresholds associated with one or more capture type classifications and/or recommended VCS pacing output settings.

In one example, the disclosure provides a medical device system including a memory configured to store a first cardiac signal sensed following delivery of a VCS pacing pulse and processing circuitry configured to receive at least the first cardiac signal and apply a pacing capture classification machine learning model to at least the first cardiac signal. The processing circuitry may be configured to determine, based on the applied pacing capture classification machine learning model, a capture type classification of the VCS pacing pulse from among a plurality of capture types. The processing circuitry may be configured to generate an output based on the capture type classification. The medical device system may include a user interface configured to, in response to the output generated by the processing circuitry, present a representation of the capture type classification associated with the delivered ventricular conduction system pacing pulse.

In another example, the disclosure provides a method including storing in a memory a cardiac signal sensed following delivery of a VCS pacing pulse, inputting at least the cardiac signal to a pacing capture classification machine learning model and outputting by the pacing capture classification machine learning model a capture type classification of the VCS pacing pulse from among a plurality of capture types. The method may further include generating by processing circuitry an output based on the capture type classification and presenting a representation of the capture type classification associated with the delivered ventricular conduction system pacing pulse by a user interface.

In yet another example, the disclosure provides a non-transitory, computer-readable storage medium storing a set of instructions which, when executed by processing circuitry of a medical device system, cause the medical device system to store in a memory of the medical device system a cardiac signal sensed following delivery of a VCS pacing pulse, apply a pacing capture classification machine learning model to at least the cardiac signal and determine, based on the applied pacing capture classification machine learning model, a capture type classification of the VCS pacing pulse from among multiple capture types. The instructions may further cause the medical device system to generate an output based on the capture type classification and present a representation of the determined capture type associated with the delivered ventricular conduction system pacing pulse by a user interface of the medical device system.

In another example, the disclosure provides a medical device system including a sensing circuit configured to sense a cardiac electrical signal, a therapy delivery circuit configured to deliver a VCS pacing pulse, and processing circuitry configured to receive the cardiac electrical signal. The processing circuitry may be configured to apply a machine learning model to at least the sensed cardiac electrical signal, the machine learning model being trained using a plurality of cardiac signal datasets to determine a pacing capture type. The processing circuitry may be further configured to determine, based on the applied machine learning model, a pacing capture type of the VCS pacing pulse. The medical device system may further include a user interface configured to present a representation of the determined pacing capture type of the VCS pacing pulse.

In another example, the disclosure provides a medical device system including a sensing circuit configured to sense at least one cardiac electrical signal, a therapy delivery circuit configured to deliver VCS pacing pulses and a memory configured to store a cardiac signal segment sensed by the sensing circuit during at least a post-pace time interval following delivery of a first VCS pacing pulse delivered by the therapy delivery circuit. The medical device system further includes processing circuitry that may be configured to apply a machine learning model to at least the first cardiac signal segment and determine, based on the machine learning model, a pacing capture type of the first VCS pacing pulse from among a plurality of capture types. The processing circuitry may select an operating pacing pulse output based on at least the determined pacing capture type. The therapy delivery circuit may be further configured to deliver a second VCS pacing pulse according to the selected operating pacing pulse output.

Further disclosed herein is the subject matter of the following examples:

Example 1. A medical device system including a memory configured to store a first cardiac signal sensed following delivery of a VCS pacing pulse and processing circuitry configured to receive the first cardiac signal sensed following delivery of the VCS pacing pulse. The processing circuitry is further configured to apply a pacing capture classification machine learning model to at least the first cardiac signal and determine, based on the applied pacing capture classification machine learning model, a capture type classification of the VCS pacing pulse from among a plurality of capture types. The processing circuitry may generate an output based on the capture type classification. The medical device system may include a user interface configured to, in response to the generated output, present a representation of the capture type classification associated with the delivered ventricular conduction system pacing pulse.

Example 2. The medical device system of example 1, wherein the memory is further configured to store a first template beat signal corresponding to a first VCS pacing pulse output. The processing circuitry may be further configured to input to the pacing capture classification machine learning model at least the first template beat signal and the first cardiac signal; and determine the capture type classification based on the pacing capture classification machine learning model applied to at least the first template beat signal and the first cardiac signal.

Example 3. The medical device system of any of examples 1-2 wherein the memory is further configured to store a first template beat signal corresponding to a first VCS pacing pulse output. The processing circuitry is further configured to determine a first template difference signal from the first cardiac signal and the first template beat signal, input at least the first template difference signal and the first cardiac signal to the pacing capture classification machine learning model; and determine the capture type classification based on the pacing capture classification machine applied to at least the first template difference signal and the first cardiac signal model.

Example 4. The medical device system of any of examples 1-3 wherein the memory is further configured to store a plurality of template beat signals, where each template beat signal corresponds to one VCS pacing pulse output of a plurality of VCS pacing pulse outputs. The processing circuitry can be further configured to input each of the plurality of template beat signals and the first cardiac signal to the pacing capture classification machine learning model and determine the capture type classification based on the pacing capture classification machine learning model applied to at least the plurality of template beat signals and the first cardiac signal.

Example 5. The medical device system of any of examples 1-4 wherein the memory is further configured to store multiple template beat signals, where each template beat signal corresponds to one VCS pacing pulse output of multiple VCS pacing pulse outputs. The processing circuitry can be further configured to determine multiple template difference signals by determining a template difference signal from each one of the multiple template beat signals and the first cardiac signal. The processing circuitry may input the plurality of template difference signals and the first cardiac signal to the pacing capture classification machine learning model and determine the capture type classification based on the pacing capture classification machine learning model applied to at least the multiple template difference signals and the first cardiac signal.

Example 6. The medical device of any of examples 4-5 wherein the memory is further configured to store the multiple template beat signals by storing a first template beat signal corresponding to a first VCS pacing pulse output, a second template beat signal corresponding to a second VCS pacing pulse output that is less than the first VCS pacing pulse output, and a third template beat signal corresponding to a third VCS pacing pulse output that is intermediate the first and second VCS pacing pulse outputs.

Example 7. The medical device system of any of examples 1-6 wherein the processing circuitry can be further configured to determine a derivative signal from the first cardiac signal, input the first cardiac signal to the pacing capture classification machine learning model by inputting at least the derivative signal, and determine the capture type classification based on the pacing capture classification machine learning model applied to at least the derivative signal.

Example 8. The medical device system of any of examples 1-7 wherein the memory is further configured to store a second cardiac signal sensed following delivery of the VCS pacing pulse, wherein the first cardiac signal being sensed by a first sensing electrode vector and the second cardiac signal being sensing by a second sensing electrode vector different than the first sensing electrode vector. The processing circuitry may be further configured to input each of the first cardiac signal and the second cardiac signal to the pacing capture classification machine learning model and determine the capture type classification based on the pacing capture classification machine learning model applied to at least the first cardiac signal and the second cardiac signal.

Example 9. The medical device system of any of examples 1-8 wherein the memory is further configured to store a pacing pulse output of the delivered VCS pacing pulse. The processing circuitry may be further configured to input at least the pacing pulse output and the first cardiac signal to the pacing capture classification machine learning model and determine the capture type classification based on the pacing capture classification machine learning model applied to at least the pacing pulse output and the first cardiac signal.

Example 10. The medical device system of any of examples 1-9 wherein the processing circuitry is further configured to determine a feature of the first cardiac signal, input the feature of the first cardiac signal and the first cardiac signal to the pacing capture classification machine learning model, and determine the capture type classification based on the pacing capture classification machine learning model applied to at least the feature of the first cardiac signal and the first cardiac signal.

Example 11. The medical device system of any of examples 1-10 wherein the processing circuitry is further configured to receive training cardiac signal datasets obtained from multiple patients, the training cardiac signal datasets including multiple training cardiac signals each sensed following delivery of a VCS pacing pulse, wherein the VCS pacing pulses associated with the multiple training cardiac signals include VCS pacing pulses delivered at multiple, different pacing pulse outputs. The processing circuitry may be configured to train the pacing capture classification machine learning model with the training cardiac signal datasets according to a machine learning algorithm and apply the pacing capture classification machine learning model trained with the training cardiac signal datasets to at least the first cardiac signal.

Example 12. The medical device system of any of examples 1-11 wherein the processing circuitry is further configured to receive multiple cardiac signals including the first cardiac signal, each of the multiple cardiac signals associated with a VCS pacing pulse, wherein the VCS pacing pulses associated with the multiple cardiac signals include VCS pacing pulses delivered at multiple different pacing pulse outputs. The processing circuitry may be further configured to input each of the multiple cardiac signals to the pacing capture classification machine learning model, determine a capture type classification of each of the VCS pacing pulses delivered at the multiple different pacing pulse outputs associated with the multiple cardiac signals based on the pacing capture classification machine learning model and determine a capture threshold for at least one capture type of the multiple capture types based on the capture type classifications.

Example 13. The medical device system of example 12 wherein the user interface is further configured to present the capture threshold for the at least one capture type.

Example 14. The medical device system of any of examples 12-13 wherein the processing circuitry is further configured to determine an operating pacing pulse output based on the capture threshold determined for the at least one capture type.

Example 15. The medical device system of example 14 further including a therapy delivery circuit configured to generate VCS pacing pulses according to the operating pacing pulse output.

Example 16. The medical device system of any of examples 1-15 wherein the processing circuitry is further configured to determine the capture type classification as one of selective His-Purkinje system capture without ventricular myocardial capture, non-selective His-Purkinje system capture with ventricular myocardial capture, ventricular myocardial capture without His-Purkinje system capture, or loss of capture.

Example 17. A method including storing in a memory a first cardiac signal sensed following delivery of a ventricular conduction system (VCS) pacing pulse and receiving by processing circuitry the first cardiac signal sensed following delivery of the VCS pacing pulse. The method may further include applying a pacing capture classification machine learning model to at least the first cardiac signal and determining, based on the applied pacing capture classification machine learning model, a capture type classification of the VCS pacing pulse from among a plurality of capture types. The method may include generating by the processing circuitry an output based on the capture type classification and presenting by a user interface a representation of the capture type classification associated with the delivered VCS pacing pulse in response to the generated output.

Example 18. The method of example 17 further comprising storing in the memory a first template beat signal corresponding to a first VCS pacing pulse output, inputting to the pacing capture classification machine learning model at least the first template beat signal and the first cardiac signal and determining the capture type classification based on the pacing capture classification machine learning model applied to at least the first template beat signal and the first cardiac signal.

Example 19. The method of any of examples 17-18 further comprising storing in the memory a first template beat signal corresponding to a first VCS pacing pulse output, determining by the processing circuitry a first template difference signal from the first cardiac signal and the first template beat signal, inputting at least the first template difference signal and the first cardiac signal to the pacing capture classification machine learning model, and determining the capture type classification based on the pacing capture classification machine learning model applied to at least the first template difference signal and the first cardiac signal.

Example 20. The method of any of examples 17-19 further comprising storing in the memory a plurality of template beat signals, where each template beat signal corresponds to one VCS pacing pulse output of a plurality of VCS pacing pulse outputs. The method may further include inputting each of the plurality of template beat signals and the first cardiac signal to the pacing capture classification machine learning model and determining the capture type classification based on the pacing capture classification machine learning model applied to at least the plurality of template beat signals and the first cardiac signal.

Example 21. The method of any of examples 17-20 further comprising storing in the memory a plurality of template beat signals, where each template beat signal corresponds to one VCS pacing pulse output of a plurality of VCS pacing pulse outputs. The method may further include determining a plurality of template difference signals by determining a template difference signal from each one of the plurality of template beat signals and the first cardiac signal, inputting the plurality of template difference signals and the first cardiac signal to the pacing capture classification machine learning model, and determining the capture type classification based on the pacing capture classification machine learning model applied to at least the plurality of template difference signals and the first cardiac signal.

Example 22. The method of any of examples 20-21 further comprising storing the plurality of template beat signals by storing a first template beat signal corresponding to a first VCS pacing pulse output, a second template beat signal corresponding to a second VCS pacing pulse output that is less than the first VCS pacing pulse output, and a third template beat signal corresponding to a third VCS pacing pulse output that is intermediate the first and second VCS pacing pulse outputs.

Example 23. The method of any of examples 17-22 further comprising determining a derivative signal from the first cardiac signal, inputting the first cardiac signal to the pacing capture classification machine learning model by inputting at least the derivative signal, and determining the capture type classification based on the pacing capture classification machine learning model applied to at least the derivative signal.

Example 24. The method of any of examples 17-23 further comprising storing a second cardiac signal sensed following delivery of the ventricular conduction system (VCS) pacing pulse, wherein the first cardiac signal being sensed by a first sensing electrode vector and the second cardiac signal being sensing by a second sensing electrode vector different than the first sensing electrode vector. The method may further include inputting each of the first cardiac signal and the second cardiac signal to the pacing capture classification machine learning model and determining the capture type classification based on the pacing capture classification machine learning model applied to at least the first cardiac signal and the second cardiac signal.

Example 25. The method of any of examples 17-24 further comprising storing in the memory a pacing pulse output of the delivered VCS pacing pulse, inputting at least the pacing pulse output and the first cardiac signal to the pacing capture classification machine learning model, and determining the capture type classification based on the pacing capture classification machine learning model applied to at least the pacing pulse output and the first cardiac signal.

Example 26. The method of any of examples 17-25 further comprising determining a feature of the first cardiac signal, inputting the feature of the first cardiac signal and the first cardiac signal to the pacing capture classification machine learning model and determining the capture type classification based on the pacing capture classification machine learning model applied to at least the feature of the first cardiac signal and the first cardiac signal.

Example 27. The method of any of examples 17-26 further comprising receiving by the processing circuitry training cardiac signal datasets obtained from a plurality of patients. The training cardiac signal datasets may include multiple training cardiac signals each sensed following delivery of a VCS pacing pulse where the VCS pacing pulses associated with multiple training cardiac signals include VCS pacing pulses delivered at multiple different pacing pulse outputs. The method may further include training the pacing capture classification machine learning model with the training cardiac signal datasets according to a machine learning algorithm and applying the pacing capture classification machine learning model trained with the training cardiac signal datasets to at least the first cardiac signal.

Example 28. The method of any of examples 17-27 further including receiving by the processing circuitry multiple cardiac signals including the first cardiac signal, each of the multiple cardiac signals associated with a VCS pacing pulse, wherein the VCS pacing pulses associated with the multiple cardiac signals include VCS pacing pulses delivered at a plurality of different pacing pulse outputs. The method may further include inputting each of the multiple cardiac signals to the pacing capture classification machine learning model, determining a capture type classification of each of the VCS pacing pulses delivered at the multiple different pacing pulse outputs associated with the multiple cardiac signals based on the pacing capture classification machine learning model and determining a capture threshold for at least one capture type of the multiple capture types based on the capture type classifications.

Example 29. The method of example 28 further including presenting by the user interface the capture threshold for the at least one capture type.

Example 30. The method of any of examples 28-29 further including determining an operating pacing pulse output based on the capture threshold determined for the at least one capture type.

Example 31. The method of example 30 further including generating VCS pacing pulses according to the operating pacing pulse output.

Example 32. The method of any of examples 17-31 further including determining the capture type classification as one of selective His-Purkinje system capture without ventricular myocardial capture, non-selective His-Purkinje system capture with ventricular myocardial capture, ventricular myocardial capture without His-Purkinje system capture, or loss of capture.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a conceptual diagram of a medical device system including an implantable medical device (IMD) capable of pacing and sensing in a patient's heart.

FIG. 1B is a conceptual diagram of an IMD connected to a pacing lead that is advanced to an alternative location within the heart for delivering VCS pacing pulses and sensing cardiac electrical signals.

FIG. 1C is a conceptual diagram of an IMD connected to a pacing lead advanced to another implant position within the heart for delivering VCS pacing pulses according to another example.

FIG. 2A is a conceptual diagram of a leadless pacemaker positioned within the RA for providing ventricular pacing via the His-Purkinje system.

FIG. 2B is a conceptual diagram of the leadless pacemaker shown in FIG. 2A implanted in an alternative location for pacing the VCS.

FIG. 3 is a schematic diagram of circuitry that may be enclosed within an IMD configured to sense cardiac electrical signals and perform VCS pacing.

FIG. 4 is a conceptual diagram of a processing circuit configured to analyze a cardiac signal for determining a pacing capture classification of the cardiac signal and an associated VCS pacing pulse according to some examples.

FIG. 5 is a flow chart of a method performed by processing circuitry of a medical device system for classifying a VCS pacing pulse and corresponding post-pace QRS waveform according to capture type in some examples.

FIG. 6A and FIG. 6B are diagrams of a PCC configured to receive multiple unknown signal inputs for each VCS pacing pulse to be classified according to some examples.

FIG. 7 is a flow chart of a method that may be performed for classifying VCS pacing pulses by processing circuitry of a medical device system according to another example.

FIG. 8 is a conceptual diagram of a processing circuit configured to analyze cardiac signals for determining a pacing capture classification according to another example

FIG. 9 is a conceptual diagram of a processing circuit configured to analyze cardiac signals for determining a pacing capture classification according to another example.

FIG. 10 is a flow chart of a method for determining a VCS pacing capture type according to another example.

FIG. 11 is a conceptual diagram of a processing circuit configured to analyze cardiac signals for determining a pacing capture classification according to yet another example.

FIG. 12 is a conceptual diagram of a processing circuit configured to analyze cardiac signals for determining a pacing capture classification according to another example.

FIG. 13 is a flow chart of a method for generating VCS pacing pulse capture type classifications during a pacing capture threshold test according to some examples.

FIG. 14 is a conceptual diagram of a graphical user interface displaying cardiac electrical signals and a marker channel annotated by capture type classifications output by a PCC according to one example.

FIG. 15 is a flow chart of a VCS capture monitoring method that may be performed by processing circuitry of a medical device system according to some examples.

### DETAILED DESCRIPTION

In general, the disclosure is directed to devices, systems and methods for classifying the type of cardiac capture achieved by a pacing pulse delivered to or in the vicinity of the His-Purkinje system. Examples of a medical device system are disclosed herein for receiving cardiac electrical signals by a cardiac signal analyzer that processes the cardiac electrical signals for classifying the signals according to capture type. The cardiac signal analyzer may be implemented as processing circuitry configured to apply an artificial intelligence or machine learning model to cardiac electrical signals to classify the type of capture associated with a cardiac pacing pulse delivered to the His-Purkinje conduction system of a patient's heart. The cardiac signal analyzer may be a machine learning model implemented in processing circuitry of a medical device system and trained using cardiac electrical signals obtained from a population of patients during His-Purkinje system pacing. An established artificial intelligence or machine learning model, e.g., a neural network model, of the trained cardiac signal analyzer receives at least one cardiac electrical signal sensed following a pacing pulse delivered to or in the vicinity of a portion of the His-Purkinje system of the patient's heart and outputs a capture type classification of the delivered pacing pulse. The cardiac signal analyzer is also referred to herein as a “pacing capture classifier” (PCC) because the cardiac signal analyzer can be implemented as processing circuitry that is programmed or trained to analyze and classify a cardiac signal according to a type of capture achieved by a delivered VCS pacing pulse.

A medical device may be configured to generate pacing pulses for delivery to the His-Purkinje conduction system of a patient's heart, e.g., according to any of the examples described herein. As used herein, the term “ventricular conduction system” (VCS) which may be used to refer to “VCS pacing.” “VCS pacing pulses,” “VCS capture,” etc., may refer to any portion of the His-Purkinje conduction system, which includes the His bundle, right and left Purkinje bundle branches and the Purkinje fibers, such that “VCS pacing” may refer generally to pacing anywhere along the His-Purkinje conduction system, and “VCS pacing pulses” refers to pacing pulses delivered anywhere along the His-Purkinje conduction system. “VCS capture” refers to capture of any portion of the His-Purkinje conduction system, which may be capture at or inferior to the His bundle, e.g., along a left and/or right bundle branch, and is also referred to herein as “His-Purkinje system capture.”

A cardiac tissue is “captured” by a pacing pulse having sufficient electrical energy to cause depolarization of the cardiac tissue at the pacing site, causing an electrical “evoked response.” The pacing-evoked depolarization at the pacing site is subsequently conducted through the heart's natural conduction system and/or myocardial tissue resulting in mechanical contraction of the heart chamber(s). In order to effectively capture and pace the heart to achieve a desired therapeutic effect, cardiac pacing pulses need to have a pulse energy that is equal to or greater than the capture threshold of the cardiac tissue at the pacing site. A pacing capture threshold test may be performed to determine the minimum pacing pulse amplitude for a given pacing pulse width (or vice versa) that captures the heart chamber. Determination of the capture threshold enables proper programming of the pacing pulse amplitude and pulse width, collectively referred to herein as the “pacing pulse output,” to promote effective pacing and avoid loss of capture. Capture monitoring by a medical device configured to deliver VCS pacing may be performed at least periodically to allow adjustments to the pacing pulse amplitude and/or pulse width to a suprathreshold value (greater than the capture threshold) when loss of capture or a change in capture type is detected.

When pacing pulses are delivered by electrodes positioned in the heart to pace the VCS, it may be possible to capture only a portion of the His-Purkinje system, e.g., complete or partial capture of the His bundle or complete or partial capture of one or both of the right and left bundle branches, without capturing ventricular myocardial tissue. In other instances, a VCS pacing pulse may capture both a portion of the His-Purkinje system and surrounding ventricular myocardium, and at other times a VCS pacing pulse may capture the surrounding ventricular myocardium without capturing any portion of the His-Purkinje system.

Capture of only the His-Purkinje system without ventricular myocardial capture is referred to herein as “selective” His-Purkinje system (SHP) capture. Capture of at least a portion of the His-Purkinje system and surrounding ventricular myocardial tissue is referred to herein as “non-selective” His-Purkinje system (NSHP) capture. Capture of the surrounding ventricular myocardium without capturing the His-Purkinje system can be referred to as ventricular myocardial only (VMO) capture. When the pacing pulse energy is below both the His-Purkinje system capture threshold and the VM capture threshold, a loss of capture (LOC) occurs. The VCS pacing pulse does not cause depolarization of cardiac cells and an evoked response does not occur.

As disclosed herein, the PCC, which may include a machine learning model, may receive multiple channels of cardiac signal data including at least one cardiac electrical signal sensed following delivery of a VCS pacing pulse of unknown capture type. The PCC is trained to output a capture type classification of the delivered VCS pacing pulse. The output capture type classification may be used for a variety of monitoring and/or pacing control functions. For example, the output capture type may be used to annotate a display of a VCS pacing pulse markers and sensed cardiac signal(s). The output capture type may be used for determining a pacing capture threshold for a given capture type and/or selecting a pacing pulse amplitude and/or pulse width for delivering VCS pacing to the patient to promote a high likelihood of achieving a desired capture type, e.g., SHP capture or NSHP capture.

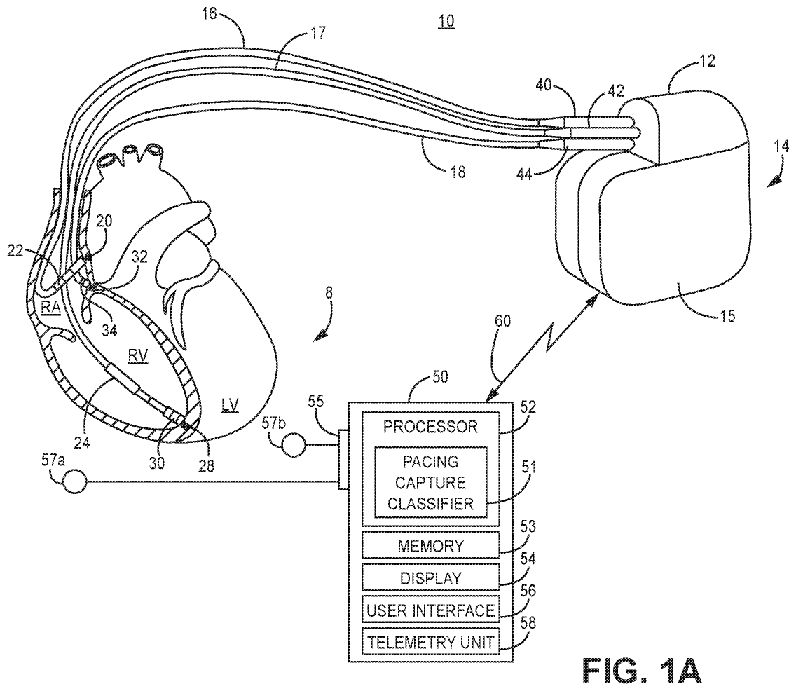

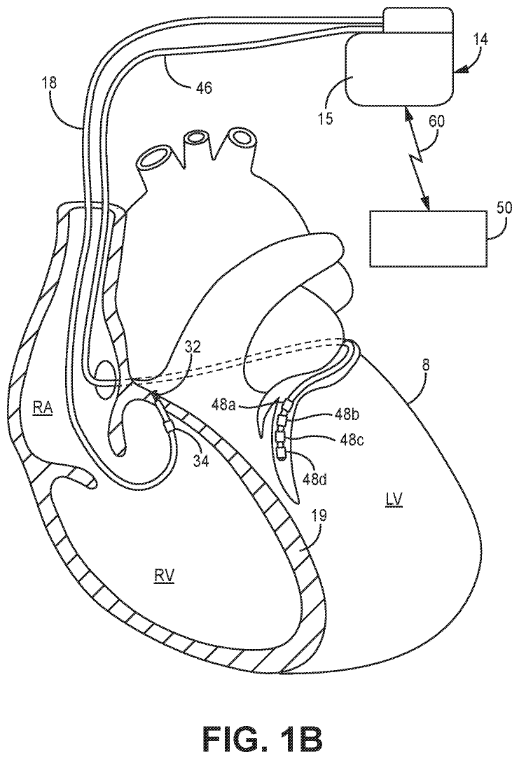

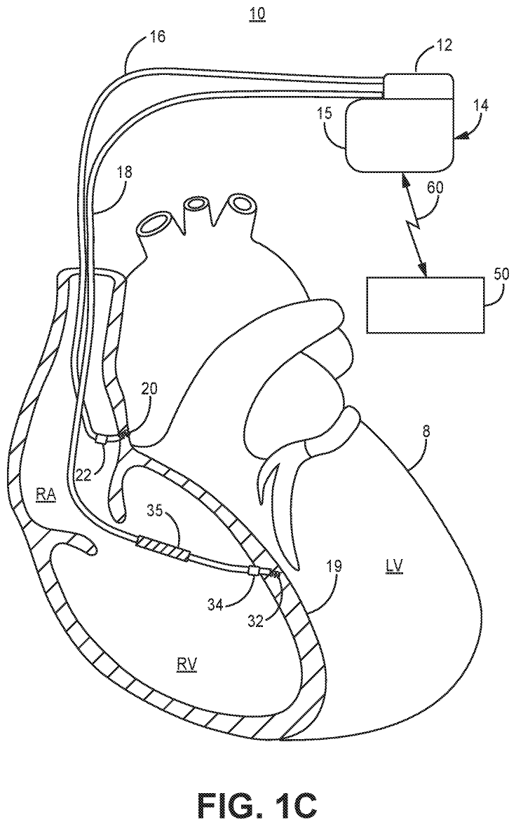

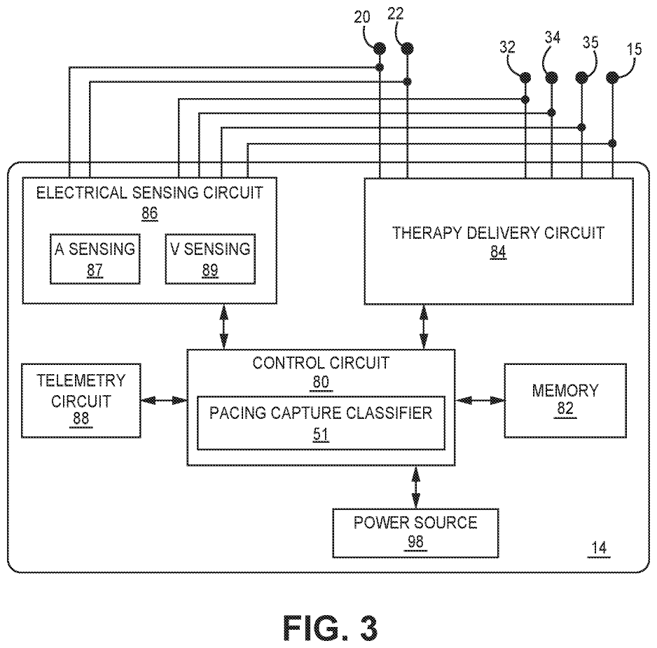

FIG. 1A is a conceptual diagram of a medical device system 10 capable of pacing and sensing in a patient's heart 8. The system 10 includes implantable medical device (IMD) 14 coupled to a patient's heart 8 via transvenous electrical leads 16, 17 and 18. IMD 14 is shown as a dual chamber device capable of delivering cardiac pacing pulses and sensing cardiac electrical signals from the right atrium (RA) and from the ventricular chambers. Housing 15 encloses internal circuitry corresponding to the various circuits and components, e.g., described in conjunction with FIG. 3 below, for sensing cardiac signals from heart 8 and delivering cardiac pacing therapy. In some examples, IMD 14 is capable of detecting tachyarrhythmias from the sensed cardiac signals and delivering high voltage cardioversion/defibrillation (CV/DF) shocks to the patient's heart 8, e.g., for terminating a detected ventricular tachycardia or ventricular fibrillation.

IMD 14 includes a connector block 12 that may be configured to receive the proximal ends of a RA lead 16 and a VCS pacing lead 18, which are advanced transvenously for positioning electrodes for sensing cardiac electrical signals and delivering pacing therapy. RA lead 16 is positioned such that its distal end is in the vicinity of the right atrium. RA lead 16 carries pacing and sensing electrodes 20 and 22, shown as a tip electrode 20 and a ring electrode 22 spaced proximally from tip electrode 20. The electrodes 20 and 22 provide sensing and pacing in the RA and are each connected to a respective insulated conductor extending within the elongated body of RA lead 16. Each insulated conductor is coupled at its proximal end to a connector carried by proximal lead connector 40. Connector block 12 is configured to receive lead connector 40 for electrically coupling conductors extending from the distal electrodes 20 and 22 to circuitry within housing 15 via electrical feedthroughs crossing housing 15.

VCS lead 18 may be advanced within the right atrium to position electrodes 32 and 34 for pacing and sensing in the vicinity of the VCS, e.g., at or near the His bundle, from a right atrial approach, as shown. VCS tip electrode 32 may be a helical electrode that is advanced into the inferior end of the interatrial septum, beneath the AV node and near the tricuspid valve annulus to position tip electrode 32 in, along or proximate to the His bundle. A ring electrode 34 spaced proximally from tip electrode 32 may be used as the return electrode with the cathode tip electrode 32 for pacing the right and left ventricles via the native His-Purkinje conduction system.

An intracardiac electrogram (EGM) signal may be produced by cardiac electrical signal sensing circuitry included in IMD 14 from the cardiac electrical signal received via a sensing electrode vector that may include tip electrode 32 and/or ring electrode 34 of VCS lead 18. The electrodes 32 and 34 are coupled to respective insulated conductors extending within the elongated body of VCS lead 18, which provide electrical connection to the proximal lead connector 44 coupled to internal IMD circuitry via connector block 12.

Housing 15 may function as a return electrode for unipolar sensing or pacing configurations with any of the electrodes carried by leads 16 and 18. Electrodes 32 and 34 may be used in a bipolar pacing electrode pair for delivering VCS pacing pulses and for receiving a cardiac electrical signal for sensing intrinsic and pacing evoked QRS waveforms. In some examples, IMD 14 may be configured to sense a far field (FF) cardiac signal, e.g., using electrode 32 and housing 15 or using electrode 34 and housing 15, and/or a near field (NF) cardiac signal, e.g., using electrodes 32 and 34, for processing and analysis for determining a capture type. Electrodes 32 and 34 may be used in a sensing electrode vector for sensing intrinsic R-waves for use in determining a heart rhythm and a need for electrical stimulation therapy.

In addition to pacing and sensing capabilities, IMD 14 may be capable of delivering high voltage cardioversion or defibrillation (CV/DF) shocks in some examples. In this case, IMD 14 may be coupled to at least one lead carrying one or more defibrillation electrodes, which may be elongated coil electrodes used to deliver high voltage CV/DF shocks. In the example shown, a third lead 17 is shown coupled to IMD 14 with a distal end advanced into the right ventricle (RV). RV lead 17 may include a coil electrode 24 used for delivering CV/DF shocks, e.g., in combination with IMD housing 15. IMD housing 15 may function as an active electrode during CV/DF shock delivery in conjunction with coil electrode 24. RV lead 17, when present, may carry a distal tip electrode 28 and ring electrode 30 for delivering ventricular pacing pulses in the RV and/or sensing a cardiac electrical signal from the RV. The electrodes 28, 30 and 24 are each connected to a respective insulated conductor extending within the elongated body of RV lead 17. Each insulated conductor is coupled at its proximal end to a connector carried by proximal lead connector 42. Connector block 12 is configured to receive lead connector 42 for electrically coupling conductors extending from the distal electrodes 24, 28 and 30 to circuitry within housing 15 via electrical feedthroughs crossing housing 15. In other examples, RV lead 17 is optional. When IMD 14 is implemented as an implantable cardioverter defibrillator (ICD) with CV/DF shock capabilities in addition to cardiac pacing and sensing functions, one or more CV/DF electrodes may be carried by RA lead 16 and/or VCS lead 18.

It is to be understood that although IMD 14 is described as a dual chamber pacemaker capable of sensing and pacing the RA and sensing ventricular signals and pacing the ventricular chambers via the VCS, with or without CV/DF shock capabilities. IMD 14 may be a single chamber pacing device with single chamber or dual chamber sensing. For example, IMD 14 may be coupled only to VCS lead 18 for sensing cardiac electrical signals and delivering VCS pacing pulses for at least maintaining a minimum ventricular rate. VCS lead 18 may carry additional sensing electrodes positioned within the RA for sensing a RA EGM signal when lead 18 is positioned for delivering VCS pacing pulses such that IMD 14 is capable of dual chamber (atrial P-wave and ventricular R-wave) sensing and VCS pacing, which may be atrial synchronous ventricular pacing delivered via the His-Purkinje system.

An external device 50 is shown in telemetric communication with IMD 14 by a communication link 60. External device 50 may be embodied as a programmer used in a hospital, clinic or physician's office to retrieve data from IMD 14 and to program operating parameters and algorithms in IMD 14 for controlling IMD functions. External device 50 may alternatively be embodied as a home monitor or handheld device for retrieving data from IMD 14. External device 50 may be used to program cardiac signal sensing parameters, cardiac rhythm detection parameters, cardiac pacing and CV/DF therapy control parameters used by IMD 14.

External device 50 may include a processor 52, memory 53, display unit 54, user interface 56 and telemetry unit 58. Processor 52 is configured to control external device operations and receive patient data and IMD data for analysis and/or for use in generating visual representations of the data to a clinician or other user. Processor 52 includes a processing circuit shown as PCC 51 for classifying VCS pacing pulses according to capture type based on analysis of received cardiac electrical signals as further described below. Processor 52 may process data and signals received from IMD 14 during a telemetry session via telemetry unit 58. Processor 52 may be configured to control telemetry unit 58 to transmit user-entered programming commands to IMD 14 and process data received from IMD 14 for display by display unit 54. In some examples, processor 52 may be configured to control telemetry unit 58 to transmit automatically generated programming commands or other information to IMD 14 that is generated by processor 52 based on pacing capture classifications determined by PCC 51.

Display unit 54, which may include a graphical user interface (GUI), displays data and other information to a user for reviewing IMD operation and programmed parameters and may display programmable parameters to a user for selection and programming of IMD 14. Display unit 54 may generate a display of the EGM signals received from IMD 14 and/or data derived therefrom. Display unit 54 may be configured to display a GUI including various windows, icons, user selectable menus, etc. to facilitate interaction by a user with the external device 50 and IMD 14. Display unit 54 may function as an input and/or output device using technologies including liquid crystal displays (LCD), quantum dot display, dot matrix displays, light emitting diode (LED) displays, organic light-emitting diode (OLED) displays, cathode ray tube displays, e-ink, or monochrome, color, or any other type of display capable of generating tactile, audio, and/or visual output. In some examples, display unit 54 is a presence-sensitive display that may serve as a user interface device that operates both as one or more input devices and one or more output devices.

As described below, display unit 54 may be configured to present representations of the capture type classifications made by PCC 51 associated with VCS pacing pulses. Such representations reduce the burden on a user or clinician in interpreting cardiac electrical signals during VCS pacing and simplify programming of IMD 14 in a patient-specific manner for promoting effective VCS pacing. Determining capture type of a VCS pacing pulse based on observation of cardiac signals can be challenging and requires considerable user expertise.

Accordingly, the techniques set forth herein for classifying VCS pacing pulses according to capture type provide specific improvements to the computer-related field of programming medical devices and reporting medical device-related information and data that have practical applications. For example, the use of the techniques herein may enable external device 50 to generate visualizations of cardiac electrical signal and/or VCS pacing pulse data annotated according to capture type classifications of delivered VCS pacing pulses. Such visualizations may more accurately inform a clinician or user as to how IMD 14 is expected to perform in delivering VCS pacing pulses for effectively capturing the VCS of the patient's heart. The capture type annotations or other related reporting of VCS pacing pulse capture type classifications can reduce the likelihood of human error in programming IMD operating parameters, such as VCS pacing pulse output. Furthermore, the techniques disclosed herein may reduce the complexity of programming IMD 14. As such, the techniques disclosed herein may enable a medical device, such as IMD 14, to be programmed to deliver VCS pacing that can achieve a desired type of cardiac capture in a manner that is simplified, flexible, and patient-specific.

User interface 56 may include a mouse, touch screen, keypad or the like to enable a user to interact with external device 50 to initiate a telemetry session with IMD 14 for retrieving data from and/or transmitting data to IMD 14. Telemetry unit 58 includes a transceiver and antenna configured for bidirectional communication with a telemetry circuit included in IMD 14 and is configured to operate in conjunction with processor 52 for sending and receiving data relating to IMD functions via communication link 60, which may include data relating to VCS pacing and capture type determination.

Communication link 60 may be established between IMD 14 and external device 50 using a wireless radio frequency (RF) link such as BLUETOOTH®, Wi-Fi, or Medical Implant Communication Service (MICS) or other RF or communication frequency bandwidth or communication protocols. Data stored or acquired by IMD 14, including EGM signals or associated data derived therefrom, results of device diagnostics, and histories of detected rhythm episodes, delivered therapies, or other data may be retrieved from IMD 14 by external device 50 following an interrogation command.

External device 50 may include external interface 55 including ports for electrical connection to surface ECG leads and electrodes 57a and 57b that may be coupled to a patient implanted with IMD 14. While only two ECG electrodes 57a and 57b are illustrated in FIG. 1A for the sake of clarity, it is to be understood that multiple surface ECG electrodes may be positioned on the patient as desired for recording a one, two, three or other desired number of ECG signals, which may include any of the limb leads I, II, III, aVR, aVL, aVF and/or any of the precordial V1, V2, V3, V4, V5, and V6 leads. Processor 52 may receive ECG signals for display by display unit 54 for observation by a user during VCS pacing. Processing and analysis of ECG signals by processor 52, which may be performed in combination with analysis of one or more EGM signals received from IMD 14, may be performed for predicting or classifying VCS pacing pulse capture type. Visual representations of ECG signals by display unit 54 may enable a user to confirm a patient's intrinsic conduction abnormality (when VCS pacing is not being delivered) and observe improvements in the ECG signals during VCS pacing, which may be annotated based on capture classifications output by PCC 51. Capture type classification annotations may enable a user to more readily recognize and identify improvements in the ECG signals with VCS pacing, such as narrowed QRS signals, decreased activation times between evoked QRS waveforms in the RV and in the left ventricle (LV) indicating a more synchronous depolarization of the left and right ventricles, or the disappearance of QRS abnormalities such as QRS features indicative of left bundle branch block, right bundle branch block or other ventricular conduction abnormalities.

PCC 51 may be implemented in processing circuitry of medical device system 10, e.g., in processor 52 of external device 50. PCC 51 may be trained using a machine learning algorithm to receive at least one cardiac signal input to be classified according to capture type. The cardiac signal input may be an EGM signal input transmitted from IMD 14. The EGM signal input may be sensed by IMD 14 during a post-pace time interval following delivery of a VCS pacing pulse by IMD 14. PCC 51 can receive multiple inputs, e.g., combined into multi-channel data points that may be aligned in time relative to a delivered VCS pacing pulse, for classifying a post-pace cardiac signal segment and associated VCS pacing pulse according to a pacing capture type, e.g., SHP capture, NSHP capture, VMO capture or LOC. In some examples, additional capture types that could be classified by PCC 51 may include fusion beats, partial His bundle capture, complete His bundle capture, selective left bundle branch (LBB) capture without myocardial capture, non-selective LBB capture with myocardial capture, LBB capture without right bundle branch (RBB) capture, LBB capture with RBB block, RBB capture without LBB capture, RBB capture with LBB block, bilateral bundle branch capture (selective and/or non-selective), selective RBB capture without myocardial capture, non-selective RBB capture with myocardial capture, retrograde atrial capture, cathodal capture without anodal capture, cathodal and anodal capture, or unknown capture type, with no limitation intended.

For the sake of illustration, examples presented herein refer to the classifications of SHP capture, NSHP capture, VMO capture and LOC as four possible capture type classifications that may be output by PCC 51. It is to be understood that PCC 51 may be trained to classify fewer or more than four capture types, or a different combination of possible capture types than the four listed above. PCC 51 may be trained to classify capture type using the techniques disclosed herein, which may include any of the examples of capture type classifications listed above or any other types of capture or beats that may occur following a VCS pacing pulse. PCC 51 may apply artificial intelligence (AI) techniques for analyzing the input for determining a capture type. The AI techniques may include deep learning techniques such as convolutional neural networks (CNN), residual CNN, feed-forward neural network (FFNN), recurrent neural network (RNN), transformer, or other machine learning techniques such as decision tree, random forest model, or other machine learning approaches for establishing a machine learning model for classifying a delivered VCS pacing pulse according to capture type based on the post-pace, unknown cardiac signal input that is obtained during a post-pace time interval following the delivered VCS pacing pulse.

The capture classification model implemented in PCC 51 may learn from cardiac electrical signal data obtained from a population of patients using machine learning. In some examples, PCC 51 is trained to classify a post-pace EGM waveform received as an unknown EGM signal input from IMD 14 by performing the capture classification algorithm using at least one other template beat input representative of a QRS waveform following a VCS pacing pulse having a pre-selected pacing pulse output. The template beat input can be a patient specific template generated from one or more QRS waveforms acquired following VCS pacing pulses delivered at a specified pacing pulse amplitude. Various examples of input signals that PCC 51 may be trained on and may subsequently receive for generating a pacing capture classification of an unknown post-pace signal input are described below.

External device 50 may be implemented as a number of computing systems configured to receive cardiac electrical signals, which may include at least one EGM signal input received directly or indirectly from IMD 14. External device 50 may be a personal computer, a medical device programmer, a home monitor, a wearable patient monitor, mobile device such as a smart phone, laptop, tablet, personal digital assistant or the like. In some examples, external device 50 is a computing device included in a remote patient monitoring system such as a computing device included in the CARELINK™ monitoring system available from Medtronic, Inc., Dublin, Ireland.

FIG. 1B is a conceptual diagram of IMD 14 coupled to VCS lead 18 advanced to an alternative location within the heart 8 for delivering VCS pacing pulses and sensing cardiac electrical signals. In this example, the distal portion of VCS lead 18 is advanced within the right ventricle (RV) for sensing cardiac electrical signals and delivering VCS pacing pulses to or in the vicinity of the His bundle from a right ventricular approach. IMD 14 may be a single chamber device coupled only to VCS lead 18. In other examples, IMD 14 may be a dual chamber device and be coupled to RA lead 16 (shown in FIG. 1A) and VCS lead 18, to enable sensing of atrial P-waves and delivery of atrial pacing pulses and delivery of VCS pacing pulses at an AV delay from atrial events, sensed or paced, in an atrioventricular synchronous pacing mode.

In this example, the tip electrode 32 of VCS lead 18 is placed in the inter-ventricular septum 19, e.g., high along the inter-ventricular septum near the inferior portion of the His bundle. Tip electrode 32 may be paired with the return anode ring electrode 34 for delivering VCS pacing pulses and for sensing raw cardiac electrical signals, which may be processed for obtaining an NF EGM signal. A post-pace NF EGM signal may be provided as input to PCC 51 in some examples for classifying capture type using the techniques disclosed herein. The tip electrode 32 or the ring electrode 34 may be paired with IMD housing 15 for receiving a raw cardiac electrical signal that is processed to obtain an FF EGM signal that may be received as input by PCC 51 for classifying capture type using the techniques disclosed herein.

In some examples, VCS pacing may be delivered in combination with LV myocardial pacing that can be delivered via an LV lead 46 for further improvement in mechanical synchrony of the RV and LV, e.g., during cardiac resynchronization therapy (CRT). LV lead 46 may be advanced into the RA, through the coronary sinus ostium and into a cardiac vein of the LV for positioning electrodes 48a, 48b, 48c and 48d (collectively “LV electrodes 48”) along the LV myocardium for sensing ventricular electrical signals and pacing the LV myocardium. LV lead 46 is shown as a quadripolar lead carrying four electrodes 48a-d that may be selected in various bipolar pacing electrode pairs for pacing the LV myocardial tissue and for sensing LV signals. One of LV electrodes 48 may be selected in combination with pacemaker housing 15 for delivering unipolar LV myocardial pacing in some instances and/or for sensing a raw cardiac electrical signal that may be processed and analyzed as an FF EGM signal for inputting to PCC 51 for classifying VCS pacing capture type according to techniques disclosed herein.

When VCS lead 18 is positioned for delivering His bundle or bundle branch pacing of one or both bundle branches, VCS pacing may be combined with ventricular myocardial pacing of the LV (using LV lead 46) to correct an LV conduction delay and achieve electrical and mechanical synchrony of the ventricles. As such, in some examples, IMD 14 may control VCS pacing pulse delivery in combination with LV myocardial pacing pulse delivery at specified time intervals which may include an AV delay and/or an VV delay. The AV delay may control the timing of the VCS pulses and/or the LV myocardial pacing pulses relative to an atrial event, e.g., sensed P-wave or delivered atrial pacing pulse. In some examples, a VV delay may control the timing between a VCS pacing pulse delivered via VCS lead 18 and an LV myocardial pacing pulse delivered via LV lead 46.

It is to be understood that LV lead 46 is optional. In some examples, IMD 14 is coupled only to VCS lead 18 advanced into the RA or the RV for delivering VCS pacing and sensing ventricular EGM signals. In other examples, RA lead 16 shown in FIG. 1A is implanted in combination with the VCS lead 18 advanced into the RA or the RV for delivering VCS pacing in a dual chamber sensing and pacing system. External device 50 may receive one or more EGM signals from IMD 14 sensed using any available ventricular EGM sensing electrode vector for processing and analysis according to the techniques disclosed herein for classifying a VCS capture type and for displaying one or more cardiac electrical signals and/or VCS pacing pulse markers annotated according to the determined VCS capture type on external device display unit 54.

FIG. 1C is a conceptual diagram of medical device system 10 including VCS lead 18 coupled to IMD 14 according to another example. VCS leak 18 is show positioned in an alternative implant site for pacing the VCS. In FIG. 1C, IMD 14 is a dual chamber device configured to receive RA lead 16, positioned in the right atrial chamber for delivering atrial pacing pulses and sensing atrial electrical signals via electrodes 20 and 22. IMD 14 may be configured to sense intrinsic atrial P-waves and deliver atrial pacing pulses in the absence of sensed P-waves. IMD 14 may be configured to deliver atrial synchronized ventricular pacing by setting an AV delay in response to each sensed P-wave or delivered atrial pacing pulse and deliver a ventricular pacing pulse to the VCS via lead 18 upon the expiration of the AV delay.

VCS lead 18 may be advanced transvenously into the RV via the RA for positioning tip electrode 32 within the inter-ventricular septum 19, relatively lower than (inferior to) the implant position shown in FIG. 1B. When tip electrode 32 is advanced relatively superiorly within the inter-ventricular septum 19, as shown in FIG. 1B, tip electrode 32 may be positioned along the inferior portion of the His bundle for delivering pacing pulses for complete or partial capture of the His bundle. In FIG. 1C, tip electrode 32 may be advanced within the inter-ventricular septum 19 in the vicinity of a bundle branch of the His-Purkinje system, e.g., at a LBB pacing site in the area of the LBB or at a right bundle branch RBB pacing site in the area of the RBB, for delivering pacing pulses for capturing one or both bundle branches.

Tip electrode 32 may be selected as a pacing cathode electrode in combination with ring electrode 34 as the return anode electrode for pacing and capturing the LBB and/or RBB in various examples. In some instances, the pacing pulse amplitude and pulse width may be selected to achieve cathodal capture at the cathode electrode for capturing at least one bundle branch. In other instances, the pacing pulse amplitude and pulse width may be selected to achieve cathodal and anodal capture, which may capture both the LBB and the RBB concurrently to provide dual or bilateral bundle branch (BB) pacing using a single bipolar electrode pair. In other examples, either tip electrode 32 or ring electrode 34 may be selected as cathode electrode paired with housing 15 in a unipolar pacing electrode vector. Unipolar pacing may capture a single BB. In some cases, however, unipolar pacing may capture both the RBB and the LBB when a unipolar pacing pulse directly captures one bundle branch while virtual current or break excitation generated by the pacing electrode may excite the other bundle branch, potentially resulting in unipolar bilateral BB pacing, with capture of both the LBB and RBB.

While VCS lead 18 is shown carrying one pacing and sensing electrode pair, tip electrode 32 and ring electrode 34, it is to be understood that in other examples, VCS lead 18 may include multiple electrodes along its distal portion to provide one or more selectable bipolar pacing electrode vectors and/or one or more unipolar pacing electrode vectors (e.g., with housing 15) for delivering pacing pulses to one or both of the RBB and the LBB.

Furthermore, VCS lead 18 may include one or more coil electrodes, e.g., coil electrode 35, when IMD 14 is configured as an ICD capable of delivering high voltage shock therapies. Coil electrode 35 may be used in sensing electrode vectors, e.g., with either of tip electrode 32 or ring electrode 34, for sensing a ventricular EGM signal that may be analyzed by PCC 51 for classifying VCS pacing pulse capture type. Other examples of pacing lead configurations that may be positioned for pacing in the area of the LBB and/or RBB are generally disclosed in U.S. patent application Ser. No. 17/370,303 (Zhou, et al.) and in U.S. Publication No. 2019/0111270 (Zhou).

As described above, IMD 14 may communicate via wireless telemetry with external device 50. External device 50 may receive EGM signals from IMD 14 sensed using any available electrodes shown in FIG. 1C or in other examples described or shown in the accompanying drawings for processing and analysis for classifying VCS pacing pulses according to capture type and/or for display by display unit 54 according to the techniques disclosed herein.

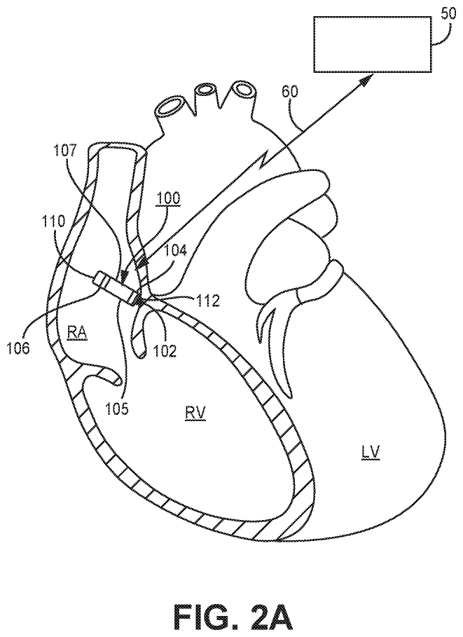

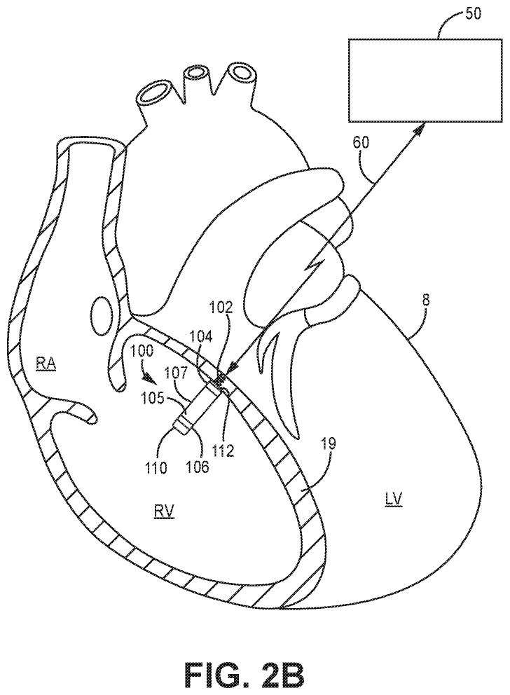

FIG. 2A is a conceptual diagram of a leadless pacemaker 100 positioned within the RA for providing ventricular pacing via the His-Purkinje system according to another example. Pacemaker 100 may include a distal tip electrode 102 extending away from a distal end 112 of the pacemaker housing 105. Pacemaker 100 is shown implanted in the RA of the patient's heart 8 to place distal tip electrode 102 for delivering pacing pulses to the His bundle. For example, the distal tip electrode 102 may be inserted into the inferior end of the interatrial septum, beneath the AV node and near the tricuspid valve annulus to position tip electrode 102 in, along or proximate to the His bundle. Distal tip electrode 102 may be a helical electrode providing fixation to anchor the pacemaker 100 at the implant position. In other examples, pacemaker 100 may include a fixation member that includes one or more tines, hooks, barbs, helices or other fixation member(s) that anchor the distal end of the pacemaker 100 at the implant site.

A portion of the distal tip electrode 102 may be electrically insulated such that only the most distal end of tip electrode 102, furthest from housing distal end 112, is exposed to provide targeted pacing at a tissue site that includes a portion of the His bundle. One or more housing-based electrodes 104 and 106 may be carried on the surface of the housing of pacemaker 100. Electrodes 104 and 106 are shown as ring electrodes circumscribing the longitudinal sidewall 107 of pacemaker housing 105. Longitudinal sidewall 107 extends from distal end 112 to proximal end 110 of housing 105. In other examples, a return anode electrode used in sensing and pacing may be positioned on housing proximal end 110. Pacing of the VCS may be achieved using the distal tip electrode 102 as the cathode electrode and either of the housing-based electrodes 104 and 106 as the return anode.

Cardiac electrical signals may be sensed by pacemaker 100 using a sensing electrode pair selected from electrodes 102, 104 and 106. For example, a cardiac electrical signal may be sensed using distal tip electrode 102 and distal housing-based electrode 104. A second cardiac electrical signal, which is a relatively more FF signal, may be sensed using electrodes 104 and 106. The raw cardiac electrical signals may be processed for producing an NF EGM signal and a relatively more FF EGM signal. The EGM signals may be used for providing input signals to PCC 51 for classifying a capture type according to the techniques disclosed herein.

In some examples, atrial P-waves may be sensed from a signal received via electrodes 104 and 106 and/or atrial pacing pulses may be delivered via electrodes 104 and 106. Atrial synchronous VCS pacing pulses may be delivered via electrodes 102 and 104 to capture at least a portion of the His-Purkinje system at an AV delay following sensed atrial P-waves and/or delivered atrial pacing pulses.

Pacemaker 100 is shown in communication with external device 50 via communication link 60. External device 50 may receive EGM signals sensed by pacemaker 100 which may be provided as input to PCC 51 for classifying a capture type of a VCS pacing pulse as further described below. Accordingly, it is to be understood that EGM signals obtained for input to PCC 51 may be sensed by electrodes carried by a lead coupled to an IMD or by electrodes carried on the housing of an IMD, which may be a leadless pacemaker configured to deliver VCS pacing or an IMD that includes housing-based electrodes for sensing cardiac electrical signals that can also be coupled to one or more leads for delivering VCS pacing pulses.

FIG. 2B is a conceptual diagram of the leadless pacemaker 100 of FIG. 2A shown implanted in an alternative location for pacing the VCS. Pacemaker 100 may be implanted within the RV along the inter-ventricular septum 19 for providing VCS pacing in some examples. Techniques disclosed herein may be used in conjunction with a leadless pacemaker, such as pacemaker 100, having a pacing electrode coupled to and extending from the pacemaker housing 105, without requiring an intervening medical lead coupled to the pacemaker for carrying the pacing and sensing electrode(s).

In this example, pacemaker 100 may be positioned within the RV for advancing the pacing tip electrode 102 extending from the distal end 112 of pacemaker housing 105 into the inter-ventricular septum 19 for pacing the VCS, e.g., in the area of an inferior portion of the His bundle or along one or both of the RBB and LBB depending on the relative positioning of tip electrode 102. Tip electrode 102 is shown as a “screw-in” helical electrode but may be configured as other types of electrodes capable of being advanced within the septal tissue. A proximal portion of the pacing tip electrode 102 may be electrically insulated, e.g., with a coating, in some examples such that only a distal portion of tip electrode 102, furthest from pacemaker housing distal end 112, is exposed to provide targeted pacing at a tissue site that includes the His bundle, LBB or RBB.

In other examples, tip electrode 102 may be formed having a straight shaft with a distal active electrode portion or other type of electrode, which may be a tissue-piercing electrode that is advanceable through the inter-ventricular septum 19 to deliver pacing, e.g., in a left portion of the septum 19 in the area of the LBB. In some examples, pacemaker 100 may include a fixation member that includes one or more tines, hooks, barbs, helices or other fixation member(s) that anchor the distal end 112 of the pacemaker 100 at the implant site and may not function as an electrode. Examples of leadless intracardiac pacemakers that may be configured for delivering cardiac pacing pulses to the His-Purkinje system that may be used in conjunction with the techniques described herein are generally disclosed in U.S. Publication No. 2019/0111270 (Zhou) and U.S. Publication No. 2019/0083800 (Yang, et al.).

Pacemaker 100 may include the distal housing-based ring electrode 104 along or near the distal end 102 of pacemaker housing 105. Distal housing-based ring electrode 104 may be selectable as the return anode electrode with tip electrode 102 for bipolar pacing of the LBB or RBB in the vicinity of the tip electrode 102. Bipolar bilateral BB pacing of both the RBB and LBB simultaneously may be achieved by cathodal capture of the LBB at tip electrode 102 and anodal capture of the RBB by distal ring electrode 104. The polarities of the tip electrode 102 and the distal ring electrode 104 may be reversed to achieve cathodal capture of the RBB and anodal capture of the LBB in some examples. Distal ring electrode 104 is shown as a ring electrode circumscribing a distal portion of the housing 105 but may alternatively be a distal housing-based electrode in the form of a button electrode, hemispherical electrode, segmented electrode or the like and may be along the face of distal end 112 of housing 105 and/or along longitudinal sidewall 107.

In the example shown, a housing-based proximal ring electrode 106, which may circumscribe all or a portion of the longitudinal sidewall 107 of the housing 105, may be provided as a return anode electrode. In other examples, a return anode electrode used in sensing and pacing may be positioned on housing proximal end 110 and may be a button, ring or other type of electrode. VCS pacing of the LBB may be achieved using the tip electrode 102 as the cathode electrode and the proximal ring electrode 106 as the return anode. Pacing of the RBB and/or myocardial tissue of inter-ventricular septum 19 may be achieved using the distal ring electrode 104 as a cathode electrode and the proximal ring electrode 106 as the return anode. In this way, bilateral or dual bundle branch pacing may be achieved using two different bipolar pacing electrode vectors carried by housing 105.

Cardiac electrical signals produced by heart 8 may be sensed by pacemaker 100 using electrodes 102. 104 and/or 106. The cardiac electrical signal received via electrodes 102 and 104, electrodes 104 and 106 and/or electrodes 102 and 106, for example, may be sensed by pacemaker 100 and processed by processing circuitry of IMD 14 and/or transmitted wirelessly, e.g., as EGM signals, to external device 50 via communication link 60. The signals may then be displayed and/or further processed and analyzed by the processor 52 of external device 50 for providing a user with visual representation of sensed EGM signals. As described herein EGM signals sensed by pacemaker 100 may be provided as input to PCC 51 for classifying a VCS pacing pulse according pacing capture type and may be displayed by external device display unit 54 with pacing capture type annotations.

The examples of FIGS. 1A-2B present various lead and/or electrode configurations that may be implemented for delivering VCS pacing in a system configured to perform the techniques disclosed herein for analyzing sensed cardiac electrical signals for classifying VCS pacing pulses according to capture type. The particular lead and electrode configurations described and shown in the accompanying drawings are intended to be illustrative in nature. It is to be understood that the leads and electrodes illustrated in FIGS. 1A-2B may be implanted in different combinations than the example combinations shown and some leads and/or electrodes may be omitted or more electrodes may be carried by a given lead or IMD housing than in the examples shown. In some examples, a leadless IMD. e.g., IMD 100, may be implanted in a patient for VCS pacing in combination with another implanted IMD, e.g., an IMD connected to a RA lead for pacing and sensing in the right atrium. A variety of lead-based and leadless IMD configurations may be conceived for sensing cardiac electrical signals and delivering VCS pacing pulses which may be used in conjunction with the techniques disclosed herein for classifying a VCS pacing pulse and associated post-pace cardiac electrical signal(s) according to one of multiple possible capture types.

FIG. 3 is a schematic diagram of circuitry that may be enclosed within an IMD configured to sense cardiac electrical signals and perform VCS pacing. The block diagram of FIG. 3 is described with reference to IMD 14 coupled to electrodes carried by RA lead 16 and VCS lead 18, e.g., as shown in FIG. 1C, as an illustrative example. It is to be understood, however, that the functionality attributed to the various circuits and components shown in FIG. 3 for sensing cardiac signals and delivering VCS pacing may be implemented in conjunction with other lead and electrode configurations, including the leadless pacemaker 100 of FIGS. 2A and 2B or other medical devices configured to deliver VCS pacing pulses and sense cardiac electrical signals.

Housing 15 is represented as an electrode in FIG. 3 for use in cardiac electrical signal sensing and, in some examples, for delivery of cardiac electrical stimulation pulses such as unipolar pacing pulses. The electronic circuitry enclosed within housing 15 includes software, firmware and hardware that cooperatively monitor cardiac electrical signals, determine when a pacing therapy is necessary, and deliver electrical pacing pulses to the patient's heart as needed according to programmed pacing mode and pacing pulse control parameters. The electronic circuitry includes a control circuit 80, memory 82, therapy delivery circuit 84, cardiac electrical signal sensing circuit 86, telemetry circuit 88, and power source 98.

Power source 98 provides power to the circuitry of IMD 14 including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86, and 88 are to be understood from the general block diagram of FIG. 3 but are not shown for the sake of clarity. For example, power source 98 may be coupled to one or more charging circuits included in therapy delivery circuit 84 for providing the power needed to charge holding capacitors included in therapy delivery circuit 84 that are discharged at appropriate times under the control of control circuit 80 for delivering pacing pulses. Power source 98 is also coupled to components of sensing circuit 86, such as sense amplifiers, analog-to-digital converters, switching circuitry, etc. as needed for sensing cardiac electrical signals. Power source 98 may provide power to the various components and circuits of telemetry circuit 88 and memory 82 as needed, which may be under the control of control circuit 80.

The circuits shown in FIG. 3 represent functionality included in IMD 14 and may include any discrete and/or integrated electronic circuit components that implement analog and/or digital circuits capable of producing the functions attributed to IMD 14 (or pacemaker 100) herein. The various components may include an application specific integrated circuit (ASIC), an electronic circuit, a processor (shared, dedicated, or group) and memory that execute one or more software or firmware programs, a combinational logic circuit, state machine, or other suitable components or combinations of components that provide the described functionality. Providing software, hardware, and/or firmware to accomplish the described functionality in the context of any modern cardiac medical device system, given the disclosure herein, is within the abilities of one of skill in the art.

Control circuit 80 communicates, e.g., via a data bus, with therapy delivery circuit 84 and sensing circuit 86 for cooperatively sensing cardiac electrical signals and controlling delivery of cardiac electrical stimulation therapies in response to sensed cardiac event signals, e.g., P-waves attendant to atrial depolarizations and R-waves attendant to ventricular depolarizations, or the absence thereof. The available electrodes are electrically coupled to therapy delivery circuit 84 for delivering electrical stimulation pulses and/or to sensing circuit 86 for sensing cardiac electrical signals produced by the heart. Sensed cardiac electrical signals may include both intrinsic signals (such as intrinsic R-waves) produced by the heart in the absence of a pacing pulse that captures the heart and evoked response signals following a delivered pacing pulse of sufficient energy to cause capture of cardiac tissue.

Sensing circuit 86 may include two or more sensing channels for receiving raw cardiac electrical signals from two or more sensing electrode vectors. For example, a RA signal may be sensed using RA lead electrodes 20 and 22 coupled to atrial sensing (A sensing) channel 87. A ventricular signal may be sensed by ventricular sensing (V sensing) channel 89 using VCS lead electrodes 32, 34 and/or 35. In some examples, V sensing channel 89 may include multiple ventricular sensing channels for receiving raw signals from multiple sensing electrode vectors that may include at least one electrode in or proximate to the ventricular chambers. For instance V sensing channel 89 may include a NF sensing channel for receiving a raw NF signal, for example using electrodes 32 and 34 of VCS lead 18 in a bipolar sensing electrode pair. V sensing channel 89 may include an FF sensing channel for receiving a raw FF signal. For example, a raw FF signal may be received using a second electrode vector having electrodes spaced further apart than the electrodes of the NF sensing electrode vector. An FF signal may be sensed, for example, using VCS lead tip electrode 32 or ring electrode 34 paired with IMD housing 15. In some examples, V sensing channel 89 may receive a raw FF signal sensed using tip electrode 32 or ring electrode 34 paired with coil electrode 35. In other examples, an FF signal may be sensed using coil electrode 35 paired with IMD housing 15. Any other available unipolar sensing electrode pair that senses a relatively more global or FF signal than the bipolar electrode pair of tip electrode 32 and ring electrode 34 may be used for sensing a ventricular EGM signal that can be used in classifying VCS capture type, alone or in combination with the ventricular NF EGM signal. The FF sensing electrode vector may have a greater inter-electrode distance than the NF sensing electrode vector, so that an FF EGM signal produced from the raw FF signal by sensing circuit 86 can be more representative of the global activation of the ventricles, or a relatively larger myocardial mass, than the NF signal. The NF signal may be more representative of local tissue activation (depolarization) at or near the VCS pacing site.

Sensing circuit 86 may include switching circuitry for selectively coupling a sensing electrode pair from the available electrodes to a respective sensing channel of A sensing channel 87 or V sensing channel 89. Switching circuitry may include a switch array, switch matrix, multiplexer, or any other type of switching device suitable to selectively couple components of sensing circuit 86 to selected electrodes.

Each of the sensing channels 87 and 89 of sensing circuit 86 may include an input filter for receiving a raw cardiac electrical signal from a respective pair of sensing electrodes, a pre-amplifier, an analog-to-digital converter and a bandpass filter for producing a multi-bit digital cardiac electrical signal, which may be referred to as an “EGM” signal when the raw signal is sensed from within a heart chamber. A multi-bit EGM signal may be passed from sensing circuit 86 to control circuit 80 for processing and analysis and/or for transmission to external device 50 (e.g., shown in FIG. 1A) for processing and analysis and/or display on display unit 54. As described below, control circuit 80 may extract a segment of a ventricular FF EGM signal and/or NF EGM signal during a post-pace time interval following delivery of a VCS pacing pulse for use in classifying the capture type of the VCS pacing pulse. The post-pace EGM signal(s) sensed following a VCS pacing pulse may be received from an FF ventricular sensing channel of V sensing channel 89 and/or a NF ventricular sensing channel of V sensing channel 89 for providing input to the PCC 51. It is to be understood that when V sensing channel 89 includes an NF sensing channel and an FF sensing channel, or other sensing channels for sensing multiple ventricular EGM signals, each sensing channel may include the same or different filtering cut-off frequencies for passing an EGM signal to control circuit 80 in which the desired QRS signal is enhanced and other undesired cardiac signals or noise are attenuated.

Each sensing channel 87 and 89 may include cardiac event detection circuitry. which may include one or more sense amplifiers, filters, rectifiers, threshold detectors, comparators, analog-to-digital converters (ADCs), timers or other analog or digital components, for detecting cardiac electrical events. For example, an atrial event detector may be included in A sensing channel 87 for sensing intrinsic P-waves attendant to intrinsic atrial depolarizations using one or both of electrodes 20 and 22 carried by RA lead 16. A ventricular event detector may be included in V sensing channel 89 for sensing intrinsic R-waves attendant to intrinsic ventricular depolarizations using electrodes 32 and 34 carried by VCS lead 18.

A cardiac event sensing threshold, such as a P-wave sensing threshold or an R-wave sensing threshold, may be automatically adjusted by sensing circuit 86 under the control of control circuit 80, e.g., based on timing intervals and sensing threshold values determined by control circuit 80, stored in memory 82, and/or controlled by hardware, firmware and/or software of control circuit 80 and/or sensing circuit 86. The R-wave sensing threshold, for example, may be controlled to start at a starting threshold voltage following a post-ventricular blanking period then decrease according to a decay profile until reaching a minimum sensing threshold. The minimum R-wave sensing threshold may be set to a programmed sensitivity of the R-wave detection circuitry. The sensitivity, programmed to a voltage level typically in millivolts, is the lowest voltage level above which a cardiac event, e.g., a P-wave or an R-wave, can be sensed by the cardiac event detection circuitry of the respective A sensing channel 87 or V sensing channel 89.

Upon detecting a cardiac electrical event based on a sensing threshold crossing, sensing circuit 86 may produce a sensed event signal that is passed to control circuit 80. For example, an atrial event detector may produce a P-wave sensed event signal in response to a P-wave sensing threshold crossing. A ventricular event detector may produce an R-wave sensed event signal in response to an R-wave sensing threshold crossing. The sensed event signals can be used by control circuit 80 for setting pacing escape interval timers that control the basic time intervals used for scheduling cardiac pacing pulses, e.g., atrial pacing pulses and VCS pacing pulses.

Control circuit 80 may include various timers or counters for counting down an AV delay, a VV delay, an atrial lower rate interval, a ventricular lower rate interval, etc. A sensed event signal may trigger or inhibit a pacing pulse depending on the particular programmed pacing mode. For example, a P-wave sensed event signal received from sensing circuit 86 may cause control circuit 80 to inhibit a scheduled atrial pacing pulse and schedule a VCS pacing pulse at the selected AV delay. If the AV delay expires before control circuit 80 receives an R-wave sensed event signal from sensing circuit 86, therapy delivery circuit 84 may generate and deliver a VCS pacing pulse at the AV delay following the sensed P-wave and in this way deliver atrial-synchronized ventricular pacing. If an R-wave sensed event signal is received from sensing circuit 86 before the AV delay expires, the scheduled VCS pacing pulse may be inhibited. The AV delay controls the amount of time between an atrial event, paced or sensed, and a VCS pacing pulse to promote electrical and mechanical synchrony of the heart chambers.

In other instances, a ventricular lower rate interval (LRI) may be set by control circuit 80 to schedule a VCS pacing pulse following a delivered VCS pacing pulse or sensed R-wave. The LRI may correspond to a programmed ventricular lower rate or may be adjusted by control circuit 80 to deliver rate response pacing when an increase in patient activity level is detected, e.g., by an accelerometer signal or other patient activity sensor included in IMD 14 (not shown in FIG. 3). When IMD 14 is operating in a dual chamber pacing mode, e.g., a DDD mode, when a P-wave is sensed during the LRI, the VCS pacing pulse can be triggered to occur at the AV delay and the LRI is restarted upon delivery of the VCS pacing pulse. If the LRI expires without a sensed P-wave or a sensed R-wave, the VCS pacing pulse is delivered at the expiration of the LRI, and the LRI is restarted. Control circuit 80 may be configured to control therapy delivery circuit 84 to deliver VCS pacing pulses according to a variety of pacing modes and pacing therapies, which may include bradycardia pacing, post-shock pacing, anti-tachycardia pacing (ATP), cardiac resynchronization therapy (CRT), rate response pacing, etc.

Therapy delivery circuit 84 may include charging circuitry, one or more charge storage devices such as one or more holding capacitors, an output capacitor, and switching circuitry that controls when the holding capacitor(s) are charged and discharged across the output capacitor to deliver a pacing pulse to a selected pacing electrode vector coupled to the therapy delivery circuit 84. Therapy delivery circuit 84 may include one or more pacing channels. In the example of IMD 14, therapy delivery circuit 84 may include an atrial pacing channel and a ventricular pacing channel each including one or more holding capacitors, one or more switches, and an output capacitor for producing pacing pulses delivered by the respective RA lead 16 (e.g., via electrodes 20 and 22) or VCS lead 18 (e.g., via electrodes 32 and 34).

Charging of a holding capacitor to a programmed pacing voltage amplitude and discharging of the capacitor for a programmed pacing pulse width may be performed by therapy delivery circuit 84 according to control signals received from control circuit 80. For example, a pace timing circuit included in control circuit 80 may include programmable digital counters set by a microprocessor of the control circuit 80 for controlling the basic pacing time intervals associated with various single chamber and/or dual chamber pacing modes, multi-chamber pacing modes when LV lead 46 is connected to IMD 14 as shown in FIG. 1B for delivering cardiac resynchronization therapy (CRT), or for delivering ATP sequences, as examples. The microprocessor of control circuit 80 may also set the amplitude, pulse width, polarity or other characteristics of the cardiac pacing pulses, which may be based on programmed values stored in memory 82.

As indicated above, IMD 14 may be configured to detect non-sinus tachycardia and deliver ATP. When IMD 14 is configured as an ICD for detecting tachyarrhythmia and delivering CV/DF shocks, therapy delivery circuit 84 may include high voltage therapy delivery circuitry for generating high voltage shock pulses in addition to low voltage therapy circuitry for generating low voltage pacing pulses. In response to detecting ventricular tachycardia or fibrillation, control circuit 80 may control therapy delivery circuit 84 to deliver a CV/DF shock. The high voltage therapy circuitry may include high voltage capacitors and high voltage charging circuitry for generating and delivering CV/DF shock pulses using coil electrodes, e.g., coil electrode 35, carried by one or more leads coupled to IMD 14 and/or housing 15.

Control parameters utilized by control circuit 80 for sensing cardiac event signals (e.g., P-waves and R-waves) and controlling pacing therapy delivery may be programmed into memory 82 via telemetry circuit 88. Telemetry circuit 88 includes a transceiver and antenna for communicating with external device 50 (e.g., shown in FIG. 1A) using radio frequency communication or other communication protocols as described above. Under the control of control circuit 80, telemetry circuit 88 may receive downlink telemetry from and send uplink telemetry to the external device 50. In some cases, telemetry circuit 88 may be used to transmit and receive communication signals to/from another medical device implanted in the patient. Telemetry circuit 88 can transmit EGM signals, e.g., as either a continuous sampled signal or as extracted post-pace EGM segments sensed during a post-pace time interval following a delivered VCS pacing pulse, for receipt by external device 50 for processing and analysis of PCC 51 implemented in external device processor 52. In some examples, an extracted post-pace time EGM segment includes one or more sample points starting before VCS pacing pulse delivery to include the isoelectric baseline of the EGM signal just prior to VCS pacing pulse delivery.

In some examples, PCC 51 may be implemented in control circuit 80. PCC 51 may be implemented in control circuit 80 for classifying EGM signals received from sensing circuit 86 following VCS pacing pulses delivered by therapy delivery circuit 84. PCC 51 may be implemented in control circuit 80 for classifying VCS pacing pulses according to capture type based on a machine learning model applied to cardiac signals after the machine learning model (e.g., neural network or other machine learning model) has been trained in an external device, e.g., external device 50 of FIG. 1A or another computing system. In other examples, PCC 51 may be trained using signals from the patient implanted with IMD 14 during a training period. The machine learning model may be established during supervised training and/or verified by a user during a validation process after a training period.

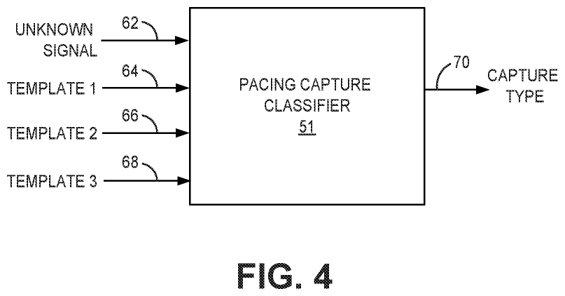

FIG. 4 is a conceptual diagram of a processing circuit configured to analyze a cardiac signal for determining a pacing capture classification of the cardiac signal and an associated VCS pacing pulse according to some examples. The processing circuit, shown as PCC 51, can include a machine learning model trained using a machine learning algorithm using multiple channels of input 62, 64, 66 and 68. PCC 51 may be implemented in external device processor 52 or another computing system, which may be a computing device included in a remote patient monitoring system or other external device. However, it is to be understood that the description of PCC 51 and various features and functions attributed to PCC 51 may be implemented in whole or in part in control circuit 80 of IMD 14.

PCC 51 is configured to receive an unknown signal input 62 that includes the post-pace cardiac electrical signal waveform to be classified as one of multiple pacing capture types. The unknown signal input 62 may be a time segment of an ECG signal sensed using surface or subcutaneous electrodes, submuscular or other extracardiac electrodes received by external device processor 52. For example, ECG signals may be received by processor 52 from ECG electrodes coupled to leads connected to external device 50 via interface 55. When an ECG signal is used as the input signal 62, the ECG sensing electrode vector may correspond to any 12 lead ECG electrode vector, which may be one of the V1-V6 ECG leads or one of V1, V2, V5 or V6 ECG leads, as examples. Processor 52 may receive the ECG signal and obtain time segment of the ECG signal that includes a post-pace QRS waveform that follows a preceding VCS pacing pulse. Processor 52 may provide the ECG signal segment to PCC 51 as the unknown signal input 62.

The unknown signal input 62 may alternatively be an EGM signal that is sensed using intracardiac electrodes. The EGM signal may be sensed by IMD 14 and transmitted by IMD telemetry circuit 88 and received by processor 52 via external device telemetry unit 58. In some instances, one or more implanted leads, such as VCS lead 18, may be coupled to external device 50 via interface 55 before connection to IMD 14, so that external device processor 52 can receive an EGM signal for obtaining the unknown signal input 62.

When input signal 62 is an EGM signal, the EGM signal may be an FF signal, which may be sensed using any available unipolar sensing electrode vector. Referring to the examples of FIGS. 1A-1C, the unknown signal input 62 may be obtained from the FF signal sensed using the VCS lead tip electrode 32 and the IMD housing 15 or the VCS lead ring electrode 34 and the IMD housing 15. When other leads and electrodes are available, another FF or unipolar sensing electrode pair may be used for sensing the EGM signal from which unknown signal input 62 is obtained. In IMD configurations that include an LV lead, e.g., as shown in FIG. 1B, the unknown signal input 62 may be obtained from an EGM signal sensed using one or more of LV lead electrodes 48 in a sensing electrode vector. For example, a unipolar sensing electrode vector may be selected using one of LV lead electrodes 48 and IMD housing 15 or one of LV lead electrodes 48 in combination with any other electrode available from RA lead 16, VCS lead 18, or RV lead 17 for sensing an EGM signal from which the unknown signal input 62 is obtained. In examples that include a CV/DF coil electrode, e.g., RV lead coil electrode 24 as shown in FIG. 1A or VCS lead coil electrode 35 shown in FIG. 1C, the unknown signal input may be obtained from an EGM signal sensed using one or more CV/DF coil electrodes in the sensing electrode vector. For instance, an EGM signal may be sensed from coil electrode 24 and housing 15 or coil electrode 35 and housing 15.

In other examples a relatively more NF EGM signal, which may be sensed using a bipolar sensing electrode vector may be received by external device processor 52 for obtaining unknown signal input 62. A NF EGM signal may be sensed using VCS lead tip electrode 32 and ring electrode 34. In the case of leadless pacemaker 100, a NV EGM signal may be sensed housing based electrodes 102, 104 and/or 106. In other examples, a bipolar EGM signal may be sensed using electrodes that may be positioned away from the VCS pacing site. For example, when LV lead 46 is available, as shown in FIG. 1B, a bipolar sensing electrode vector may be selected from any pair of electrodes 48. In still other examples, when RV lead 17 is connected to IMD 14, an EGM signal may be sensed between any of RV lead tip electrode 28 and RV lead ring electrode 30, RV lead tip electrode 28 and RV lead coil electrode 24, RV ring electrode 30 and RV lead coil electrode 24, or any of the RV lead electrodes 24, 28 or 30 and IMD housing 15.

For each VCS pacing pulse delivered for which a capture classification is desired, the unknown signal input 62 may be obtained from the cardiac electrical signal, e.g., an ECG or EGM signal, sensed over at least a predetermined post-pace time interval, e.g., including at least a predetermined number of post-pace samples. In various examples, the unknown signal input is 35 to 100 samples or between 50 and 70 samples of the ECG or EGM signal sampled at 256 Hz. The signal input 62 may be 242 milliseconds (ms) long in an illustrative example, when the sampling rate is 256 Hz and 62 samples of the ECG or EGM signal are obtained, which may include at least on sample point prior to the time point of the delivered VCS pacing pulse. In other examples, the input signal 62 may be sampled at 128 Hz or 512 Hz and the sample number may be adjusted accordingly to obtain an ECG or EGM signal input that is, for example, between 150 and 600 ms in length, between 200 and 500 ms in length, or about 200 to 350 ms in length.

The input signal 62 may be sampled starting from one or more sample points before the time point of the VCS pacing pulse delivery to capture information about the isoelectric baseline of the ECG or EGM signal, at or just prior to the time point of the VCS pacing pulse delivery. Inherent cardiac electrical signal baseline differences between different input signals received by PCC 51 can be corrected for by zeroing the first input signal sample point obtained prior to the VCS pacing pulse delivery. In other examples, sampling of the input signal 62 may begin after a delay of one or more sample points, e.g., to reduce the pacing artifact content in the unknown signal input. When the cardiac electrical signal is received directly by external device 50, e.g., via interface 55, a higher sampling rate of the input signal 62 may be utilized because processing power and burden may be less limited than when an EGM signal is received from IMD 14 or when PCC 51 is implemented in IMD 14.

The post-pace time interval that is classified by PCC 51 may extend for a predetermined time interval from the sample point of the VCS pacing pulse delivery or beginning from a predetermined delay of one or more sample points after the VCS pacing pulse. Sample points acquired prior to the VCS pacing pulse may be used only for zeroing the baseline of the input signal. As such, as used herein, the term “post-pace time interval” may not be exclusively “post-pace,” following a delivered pacing pulse, and may include one or more sample points prior to a delivered VCS pacing pulse to enable baseline zeroing and time alignment of multiple signal inputs received by PCC 51. However, it is the QRS waveform (or lack of QRS waveform) following the delivered VCS pacing pulse that is present in the post-pace time interval that is analyzed by PCC 51 for classifying the delivered VCS pacing pulse and associated post-pace waveform according to capture type.

While one unknown input signal 62 is shown in FIG. 4 that is sensed using one sensing electrode vector, it is contemplated that multiple unknown input signals may be received by PCC 51 that are sampled for at least post-pace time interval following a VCS pacing pulse for use in classifying the VCS pacing pulse according to one of multiple types of capture. For example, PCC 51 may receive one input signal sensed from an FF EGM signal sensing electrode vector and a second input signal from a NF EGM signal sensing electrode vector or an ECG signal sensing electrode vector. With reference to FIG. 1A, PCC 51 may receive a first unknown signal input sensed from an FF (e.g., unipolar) sensing electrode vector between VCS lead tip electrode 32 or VCS lead ring electrode 34 and IMD housing 15 (which may be referred to as a “tip-to-can” or “ring-to-can” sensing vector, respectively). PCC 51 may receive a second unknown signal input sensed from a NF (e.g., bipolar) sensing electrode vector between VCS lead tip electrode 32 and VCS lead ring electrode 34. In other examples, any combination or two or more of any of the example sensing electrode vectors described herein or available from the electrodes coupled to IMD 14 and/or external device 50 may be used for sensing a cardiac electrical signal that is sampled over at least a post-pace time interval for obtaining multiple unknown signal inputs received by PCC 51 for capture type classification.

In some examples, PCC 51 receives two more unknown signal inputs sampled from ECG signals such as any of the V1, V2, V3, V4, V5 and/or V6 ECG signals or any of the 12 lead ECG signals. Multiple ECG signals may be sampled over at least a post-pace time interval for providing multiple unknown signal inputs to be collectively classified along with the preceding VCS pacing pulse as one of multiple possible capture types by PCC 51. In an illustrative example, one of the V1, V2 or V3 ECG signals and one of the V4, V5 or V6 ECG signals are each sampled over a time interval including one or more sample points prior to the VCS pacing pulse and a predetermined number of sample points after the VCS pacing pulse and provided as two unknown signal inputs for capture type classification by PCC 51.

In still other examples, a combination of one or more EGM signals and one or more ECG signals may be provided as unknown input signals to be collectively classified by PCC 51. To illustrate, the FF signal sensed tip-to-can from the VCS lead tip electrode 32 to housing 15 and the V5 or V6 ECG signal (or any other selected ECG signal) may be sampled over a post-pace time interval (which may include one or more sample points prior to the delivered VCS pacing pulse) and received as unknown signal inputs by PCC 51.

In addition to receiving at least one unknown signal input 62 to be classified, PCC 51 can be configured to receive at least one template beat input. The template beat input may be sampled from a selected cardiac electrical signal during a post-pace time interval following a VCS pacing pulse delivered at a selected pacing pulse output. Three template beat inputs 64, 66 and 68 are shown for the sake of example in FIG. 4. As described below, however, one, two, three or more template beat inputs may be received as input by PCC 51. Each of the input signals received by PCC 51 may be combined into a series of multi-channel data points. In the example of FIG. 4, the unknown signal to be classified and each of the three template beat inputs may be combined into a multiple channel input as represented by inputs 62, 64, 66 and 68.

Each template beat input 64, 66 and 68 may be generated from the cardiac electrical signal sensed by the same sensing electrode vector used for obtaining the unknown signal input 62 (or one of the unknown signal inputs when multiple unknown signal inputs are received by PCC 51). Each template beat input 64, 66, and 68 may be obtained over a post-pace time interval using the same sampling rate as the unknown signal input 62. The template beat inputs 64, 66 and 68 may each correspond to a known pacing pulse output but can be an unknown capture type for the given patient. As such, the template beat inputs 64, 66, and 68 can be unknown capture types but may be established during VCS pacing at a preselected pacing pulse output, such as a maximum, minimum and/or intermediate pacing pulse output setting available from the programmable pacing pulse output settings or used during a capture threshold search. As described in examples given below, a template beat input may be established during VCS pacing at a relatively high pacing pulse amplitude, e.g., 5 volts or higher, that may be expected to capture at least a portion of the His-Purkinje conduction system and/or ventricular myocardium.

Each template beat input 64, 66 and 68 may be obtained by the IMD control circuit 80 or by external device processor 52 from a post-pace time interval following a single VCS pacing pulse delivered at a known pacing pulse output. In other examples, each template beat input may be obtained by averaging multiple individual cardiac signal segments that are each sensed over a predetermined post-pace time interval after delivery of a VCS pacing pulse delivered at the selected pacing pulse output. As described above, the sampled signal may include one or more sample points prior to the delivered VCS pacing pulse and sample points acquired over a predetermined time interval after the VCS pacing pulse such that the “post-pace” time interval includes at least a predetermined time interval following the VCS pacing pulse and may include one or more sample points prior to the VCS pacing pulse. Each template beat input 64, 66 and 68 may therefore include an equal number of one or more sample points prior to the VCS pacing pulse and the post-pace signal template to enable processor 52 to perform baseline zeroing and time alignment of the template beat inputs 64, 66 and 68.

The template beat inputs 64, 66 and 68, once established, may be stored in IMD memory 82 or external device memory 53 and retrieved by PCC 51 when an unknown signal input 62 is received for capture type classification. In other examples, the template beat inputs 64, 66 and 68 may be obtained each time a capture threshold test is performed by the medical device system by creating each template beat signal from the post-pace sensed cardiac electrical signal following each of a predetermined number of VCS pacing pulses at a respective, selected pacing pulse output.

One or more of the unknown signal input 62 and/or template beat inputs 64, 66 and 68 may include a leading (or trailing) data value. The data value may be related to a signal feature of the respective unknown signal input or template beat or related to the respective VCS pacing pulse output associated with the unknown input signal or template beat input. For example, the unknown signal input may include the sampled post-pace cardiac electrical signal prepended or appended with the pacing pulse amplitude or pacing pulse width of the delivered VCS pacing pulse to be classified. A template beat input 64, 66, or 68 may include the template beat signal obtained over the predetermined post-pace time interval (during one or more VCS paced cycles) that is prepended or appended by a data value corresponding to the pacing pulse output associated with the template beat signal. For example, a template beat input may be acquired during VCS pacing at a predetermined pacing pulse voltage amplitude, e.g., between 1 and 10 volts or between 2 and 8 volts. In one example, at least one template beat input 64, 66 or 68 is established during VCS pacing at 5 volts or a highest pacing voltage amplitude used during a capture threshold test. The value of the pacing pulse amplitude, e.g., 5 volts, may be prepended or appended to the associated template beat signal.

The prepended or appended pacing pulse output data value, e.g., the pacing pulse voltage amplitude or a pacing pulse width, may be separated from the respective unknown signal sample point values or template sample point values by zeros. Zero padding may be included before and/or after the pacing pulse output data value to separate the pacing pulse output data value from the cardiac electrical signal sample point values to avoid interaction of the pacing pulse output value and the cardiac signal sample point values during training and during VCS pacing pulse classification after the machine learning model is established. Zero padding before and after the pacing pulse output data value can avoid or reduce unwanted effects on the edge of the cardiac electrical signal of the unknown signal input or the template beat input during machine learning.

In other examples, a feature of the post-pace unknown signal to be classified and/or template beat signal may be prepended or appended to the respective input signal provided to PCC 51. For example, processor 52 (or control circuit 80) may determine a feature of the unknown signal segment to be classified and/or the template beat signal(s) to provide as a prepended or appended data value. The signal feature could be a maximum peak amplitude, an activation time determined as a time interval from the VCS pacing pulse to a maximum peak amplitude, a signal width, a signal area, a maximum positive slope, a maximum negative slope, time between a maximum positive slope and a maximum negative slope, or any other selected cardiac electrical signal feature. For instance, an LV activation time may be prepended to an input signal. The LV activation time may be determined from the time of a delivered VCS pacing pulse to the maximum peak amplitude of the post-pace QRS signal (or another fiducial feature of the QRS signal) that may be sensed from an LV sensing electrode vector, e.g., using a VCS pacing tip electrode positioned for pacing the LBB or using an electrode carried by the LV lead 46. In some examples, a first data value may be prepended to the unknown signal input and/or one or more template beat signals and a second data value may be appended to the unknown signal input and/or one or more template beat signal to provide the PCC input. The data values may include a value relating the associated VCS pacing pulse output and/or a value relating to a cardiac electrical signal feature of the respective unknown input signal or template beat input and/or patient demographic information, such as the age, sex or other patient related information. External device processor 52 or IMD control circuit 80 may determine the data values to be prepended or appended to a respective input signal.

PCC 51 can be trained using cardiac signal datasets from a population of patients using a supervised deep learning technique such as convolutional neural networks (CNN), residual CNN, feed-forward neural network (FFNN), recurrent neural network (RNN), transformer, or other machine learning techniques such as decision tree, random forest model, or other machine learning approaches to build a machine learning model, e.g., a neural network model, using the number of unknown signal inputs and template beat inputs to be received by PCC 51 for VCS pacing pulse capture type classification. Inputs received by PCC 51 during a training session are acquired in an analogous manner, e.g., using the same sensing electrode vector(s), sampling rate and post-pace time interval, as the signals that will be acquired for inputting to PCC 51 for classifying the VCS pacing pulse capture type after training is complete and the machine learning model is fixed.

A PCC training dataset may be obtained from each one of multiple patients, with each dataset including the required input signals. The multiple datasets obtained from multiple patients define an “epoch” that may be input to PCC 51 multiple times during a training session. Training may be complete after a fixed number of epochs, e.g., the entirety of the training datasets from the population of patients has been passed to the PCC 51 a fixed number of times. In other examples, the PCC training may be stopped once the outputs and associated confidence levels have not changed more than a specified amount for one or more recent epochs.

The trained machine learning model of PCC 51 may be validated by inputting validation datasets obtained from one or more patients. The validation datasets may be different than the training datasets but acquired in analogous manner. The validation datasets may be from a different or smaller group of patients than the training datasets. The output 70 of PCC 51 resulting from input validation datasets can be validated by expert truthing of the output capture type classifications. The training of PCC 51 may be complete when the validation of the PCC output during training and/or validation by an expert has reached a certain threshold, which may be a minimum percentage of accurate classifications and/or maximum percentage of inaccurate classifications according to capture classifications truthed by an expert.

The output 70 of PCC 51 includes at least a capture type classification of the VCS pacing pulse associated with the unknown input signal 62. The output 70 may include a level of confidence of the classified capture type, which may be expressed as a percentage. In some examples, the output 70 may include a confidence level corresponding to each of the possible capture type classifications. Processor 52 may determine a capture type classification of a delivered VCS pacing pulse by applying the machine learning model of PCC 51 to the input signal(s). Processor 52 may determine the capture type classification associated with a highest confidence level output of the applied PCC machine learning model. In some examples, if the highest level of confidence out of all capture type classifications is less than a threshold percentage, the capture type classification may be determined by processor 52 to be “unknown.”

The output 70 of PCC 51 may be used by external device processor 52 for generating one or more outputs or information relating to the VCS pacing capture classification. In some examples, processor 52 and display unit 54 may cooperatively generate a visual representation of the capture type classification, e.g., in a GUI of display unit 52. Each classified VCS pacing pulse and/or corresponding post-pace cardiac electrical signal waveform may be displayed and annotated according to the classified capture type. In some examples, the level of confidence corresponding to the classified capture type may be displayed for each classified VCS pacing pulse. As described below, the PCC output 70 may be obtained by processor 52 during a pacing capture threshold test for use in displaying annotated VCS pacing markers and/or cardiac electrical signals according to capture type. The output 70 obtained during a capture threshold test may be used by processor 52 in selecting a VCS pacing pulse output setting for achieving capture of at least a portion of the VCS or a desired type of VCS pacing capture. The selected VCS pacing pulse output setting may be displayed in a GUI of display unit 54 as a recommended setting or automatically programmed into IMD 14 via telemetry unit 58. Processor 52 may generate output for display by display unit 54 in a GUI that includes a table or graphical representation of the pacing pulse outputs delivered during a capture threshold test and the corresponding capture type classification(s) associated with each pacing pulse output.

Additionally or alternatively, the PCC output 70 may be used by external device processor 52 for determining a percentage of delivered VCS pacing pulses corresponding to each of the possible capture types for reporting in a tabular or graphical display. The report of the percentages of each type of capture type classification of VCS pacing pulses may inform a clinician for use in patient management and pacing therapy control parameter optimization. When PCC 51 is implemented in IMD control circuit 80, memory 82 may store counts of each type of capture classification so that control circuit 80 can determine a percentage of each determined type of capture associated with VCS pacing pulse delivery and the results may be transmitted to external device 50 for generating a report for display by display unit 54.

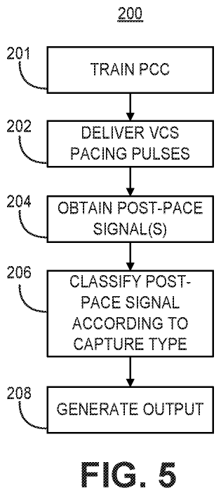

FIG. 5 is a flow chart 200 of a method performed by processing circuitry of a medical device system for classifying a VCS pacing pulse and corresponding post-pace QRS waveform according to capture type in some examples. The technique of FIG. 5 is primarily described as being performed by processor 52 of external device 50, with IMD 14 delivering VCS pacing pulses and sensing EGM signals that can be received by processor 52 through telemetric transmission between IMD 14 and external device 50. In other examples, another processor, alone or in combination with processor 52, may perform any part of the technique of FIG. 5. For example, control circuit 80 of IMD 14 or a processor of another computing device alone or in combination with processor 52, may perform any part of the techniques of FIG. 5. Input data used for training PCC 51 at block 201 may be obtained from multiple different patients, each implanted with an IMD and associated lead and electrode configuration, e.g., IMD 14 and any of the lead and electrode configurations describe above in conjunction with FIGS. 1A-1C. As such, the control circuit 80 of the IMD 14 implanted in each patient may be involved in obtaining the training data required to perform the process of FIG. 5. Each IMD delivers VCS pacing pulses at varying pacing pulse outputs and senses EGM signals from which cardiac electrical signal segments can be obtained for generating training and validation datasets used as inputs to PCC 51 at block 201 during a training session and a validation session.

In other examples, an external pacing device, which may be implemented in external device 50 or a pacing system analyzer (PSA), may be configured to generate VCS pacing pulses that may be delivered using implanted leads positioned for VCS pacing, such as VCS pacing lead 18, prior to connection to IMD 14. VCS pacing lead 18 may be electrically connected to interface 55 such that electrical pulses generated by external device 50 can be delivered as VCS pacing pulses enabling acquisition of post-pace cardiac electrical signals used for obtaining inputs for training PCC 51.

At block 201, PCC 51 is trained with training data that includes post-pace cardiac electrical signal inputs obtained from a population of patients. The post-pace cardiac electrical signal inputs may be obtained from ECG and/or EGM signals following VCS pacing pulses over a predetermined post-pace time interval and selected sampling rate, e.g., according to any of the examples described above. As indicated above, the “post-pace” cardiac electrical signal inputs may not be exclusively post-pace sample points because each signal input may include one or more pre-pace sample points used for temporal alignment and baseline zeroing.

The input training data acquired from multiple patients may be subsequently loaded into memory 53 of external device 50 for retrieval by processor 52 and provided as input to PCC 51 during a machine learning training session. In some examples, PCC 51 is trained to classify capture type using two or more unknown signal inputs that are obtained over the same time interval following a VCS pacing pulse from two or more different cardiac signal sensing electrode vectors, which may be ECG and/or EGM signal sensing electrode vectors.

In other examples, PCC 51 is trained to classify the capture type using at least one unknown signal input for each VCS pacing pulse to be classified and using one or more template beat inputs as described above in conjunction with FIG. 4 and further described below in conjunction with the accompanying flow charts and diagrams. For example, as described below, processor 50 may determine a template beat difference signal as the difference between an unknown input signal to be classified and a template beat signal established from at least one cardiac electrical signal sensed during a post-pace time interval following a VCS pacing pulse delivered at a pre-selected pacing pulse output. One or more template beat difference signals, each corresponding to a different pre-selected pacing pulse output, may be provided as input(s) to PCC 51, in combination with one or more unknown input signal(s) during the machine learning training session. In some examples, the PCC inputs additionally include one or more template beat inputs, which may or may not correspond to the template beat signals used to determine the template beat difference signals.

Typically, the training data may be obtained from a group or population of different patients. PCC 51 may operate to analyze and process received data to determine a VCS pacing pulse classification according to different capture types with a high degree of accuracy. PCC 51 may be trained under supervision where an expert confirms classified capture type based on visual inspection or analysis of input signal features. Typically, the greater the amount of input signal data on which PCC 51 is trained, the higher the accuracy of the machine learning model of PCC 51 in classifying capture type of new unknown signal inputs.

After the machine learning model (e.g., a neural network or other model) of PCC 51 is trained using the training dataset, a validation dataset, different than the training dataset, obtained from one or more patients may be input to PCC 51. An expert may confirm the trained PCC output capture type classifications based on visual inspection.

Once the machine learning model is fixed, a VCS pacing pulse may be delivered at block 202 by IMD 14 implanted in an individual patient for capture type classification. At block 204, external device processor 52 obtains the post-pace cardiac electrical signals needed for providing input to PCC 51. IMD 14 may transmit one or more EGM signals to external device 50. Processor 52 may receive the EGM signals for extracting the post-pace cardiac signal data to provide as input to PCC 51. External device processor 52 may receive an EGM signal transmitted by telemetry in real time as VCS pacing pulses are being delivered by IMD 14 and extract post-pace EGM signal segments for providing input to PCC 51. In other examples, post-pace EGM signal segments may be stored in IMD memory 82, obtained during VCS pacing, and transmitted to external device 50 for post-processing.

Processor 52 obtains at least one unknown input signal to be classified and any other data inputs required by PCC 51 per the machine learning model established during the training session. Other signal inputs are described herein in conjunction with the accompanying diagrams and flow charts and may include template beat inputs, template difference inputs, derivative signal inputs, VCS pacing pulse related data input and/or cardiac signal feature related input.

PCC 51 applies the trained model to the individual patient data at block 206 to determine a capture type classification of the delivered VCS pacing pulse. A series of VCS pacing pulses may be delivered at block 202. Processor 52 receives the required cardiac electrical signals to extract the post-pace unknown signal input for each delivered VCS pacing pulse in a series of pulses. Processor 52 may receive or determine any other required data from the individual patient cardiac signals for input to and classification by PCC 51 based on the applied machine learning model. Each delivered VCS pacing pulse in a series of (consecutive or non-consecutive) VCS pacing pulses may be classified according to capture type at block 206.

PCC 51 outputs each VCS pacing pulse capture classification for use by external device processor 52 in generating information displayed by display unit 54 and/or transmitted to IMD 14. The PCC output may include a level of confidence, e.g., as a percentage confidence, associated with the capture type classification or associated with each possible capture type including the capture type classification determined for the delivered VCS pacing pulse. The level of confidence may be used by processor 52 to further post-process the PCC output, for example to change a capture type classification to “unknown” if the level of confidence of the capture type classification having the highest level of confidence is below a predetermined threshold. The level of confidence threshold may be programmable by a user for determining, by external device processor 52, when a determined capture type classification is unknown.

At block 208, external device processor 52 may generate an output useful in managing or controlling the VCS pacing therapy, such as for automatic or user selection of VCS pacing pulse output settings. External processor 52 may generate an output based on the capture type classifications determined by PCC 51. For example, each classification and associated confidence level may be stored in external device memory 53. Each classification may be stored in association with the pacing pulse output of the classified VCS pacing pulse. However, it is to be understood that the pacing pulse output of each VCS pacing pulse to be classified may or may not be provided as input to PCC 51. The PCC output may be compiled with other VCS pacing pulse data and/or cardiac signal information by processor 52 for generating information and data for display by display unit 54 in a GUI. For example, external device processor 52 may combine the PCC output with VCS pacing pulse timing and pacing output data and with received cardiac electrical signals for generating a visual representation in a GUI of display unit 54 that includes a cardiac electrical signal sensed during VCS pacing and VCS pacing pulse markers annotated by capture type classifications output by PCC 51, which may include a notation of the associated level of confidence.

In some examples, the external device processor 52 may generate an output at block 208 based on the PCC capture type classifications that includes a capture threshold for achieving one or more types of capture by the VCS pacing pulses. External device processor 52 may be configured to perform an analysis of the capture type classifications in combination with associated pacing pulse output data received from IMD 14 for determining the lowest pacing pulse output, e.g., the lowest pacing pulse amplitude for a given pulse width, at which a particular capture type classification is made. A capture threshold may be determined by processor 52 based on a threshold level of confidence of the capture type classification. For example, a capture threshold for a given capture type may be determined to be the lowest VCS pacing pulse amplitude resulting in the given capture type classification with at least a 70%, 80%, 90% or other threshold level of confidence. One or more capture thresholds may be reported for each of one or more capture type classifications, e.g., in a GUI displayed by display unit 54. For instance, capture thresholds that may be determined by processor 52 and reported in a GUI of display unit 54 can include a SHP capture threshold, NSHP capture threshold and/or VMO capture threshold.

In still other examples, external device processor 52 may generate an output at block 208 based on the PCC capture type classifications that indicates a recommended pacing pulse output setting. Based on one or more determined capture thresholds, external device processor 52 may generate an indication of a recommended pacing pulse output, e.g., a recommended pacing pulse amplitude for a given pulse width, that is a safety margin greater than a determined capture threshold for promoting capture of at least a portion of the VCS by each VCS pacing pulse delivered by IMD 14.

The output generated by external device processor 52 at block 208 may include a communication signal transmitted to IMD 14 by external device telemetry unit 58. External device processor 52 may transmit a signal confirming VCS capture (e.g., SHP or NSHP capture) or indicating another capture type (e.g., VMO capture or LOC). IMD 14 may receive the transmitted signal and respond by maintaining VCS pacing control parameters at current values when a desired capture type is confirmed (e.g., when SHP or NSHP capture is confirmed) or adjusting the VCS pacing control parameters when an undesired capture type is confirmed (e.g., when VMO capture or LOC is indicated). Additionally or alternatively, external device processor 52 may control telemetry circuit 58 to transmit a programming command to adjust a VCS pacing pulse output setting to a value determined based on analysis of the output of PCC 51.

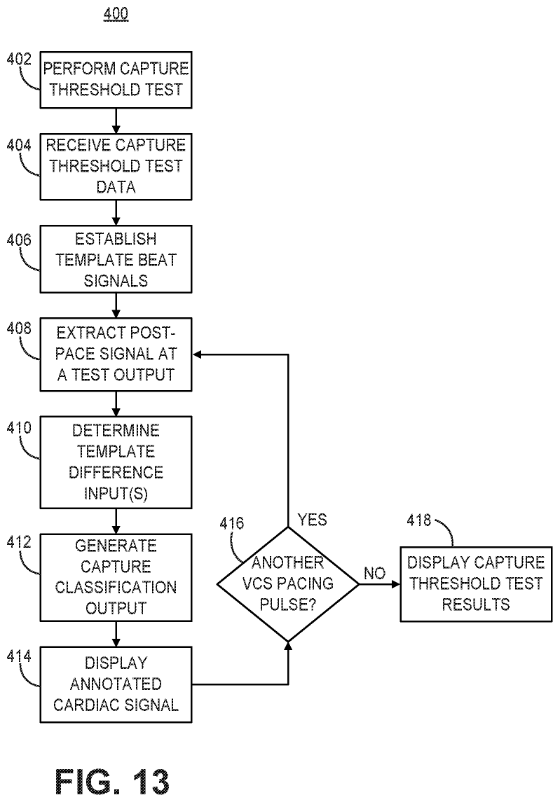

In some cases, the output generated at block 208 may include a displayed notification of a recommended change in the VCS pacing electrode vector (e.g., when more than one pacing electrode vector is available for delivering VCS pacing pulses) or a programming command transmitted to IMD 14 to change the pacing electrode vector. For instance, if SHP or NSHP capture is not confirmed based on capture classifications output by PCC 51 when the pacing pulse output is at maximum available (or maximum acceptable) pacing output, a different VCS pacing electrode vector may be selected in an attempt to deliver VCS pacing pulses that effectively capture at least a portion of the His-Purkinje system as an acceptable pacing pulse output. In other instances, when a VMO or LOC notification is received by IMD 14, IMD 14 may perform a capture threshold test, which may include classification of VCS pacing pulses delivered during the capture threshold test at different pacing pulse outputs for one or more different VCS pacing electrode vectors. Techniques for performing a capture threshold test are described below, e.g., in conjunction with FIGS. 13-15.

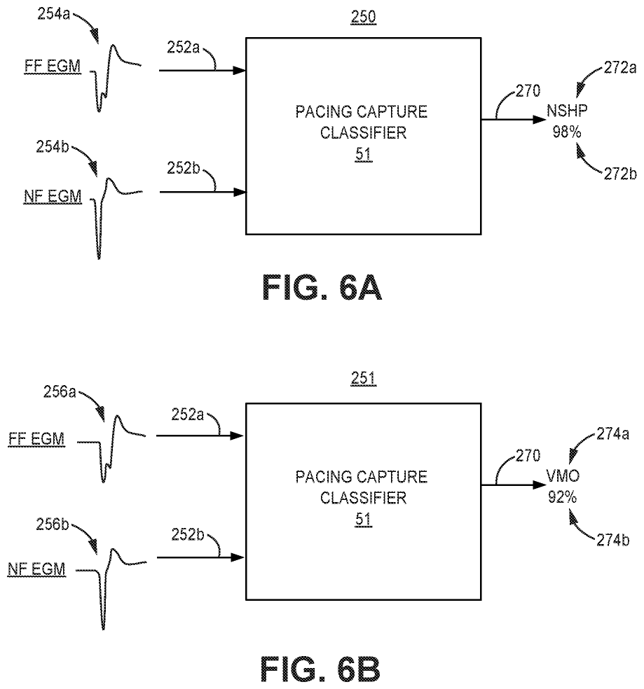

FIG. 6A and FIG. 6B are diagrams 250 and 251 of PCC 51 configured to receive multiple unknown signal inputs for each VCS pacing pulse to be classified according to some examples. In these examples, PCC 51 is configured to receive a first unknown signal input 252a obtained from an FF EGM signal and a second unknown signal input 252b obtained from a NF EGM signal. Both unknown signal inputs 252a and 252b are sensed during the same post-pace time interval following the same VCS pacing pulse of unknown capture type but using different sensing electrode vectors. One sensing electrode vector may be selected for obtaining a relatively more global or FF signal, and the second sensing electrode vector may be selected to sense a relatively more local or NF signal. PCC 51 is previously trained to output a determined capture type classification 270 by applying a machine learning model to the FF EGM and NF EGM unknown signal inputs 252a and 252b. Each of the signal inputs 252a and 252b may be time aligned starting with the first sample point of the signal input, which may be a sample point prior to the VCS pacing pulse. The baseline may be zeroed for each unknown signal input 252a and 252b by zeroing the first, pre-pace sample point of each signal segment.

In FIG. 6A, the two EGM signal segments 254a and 254b are received as first and second unknown signal inputs 252a and 252b and classified by PCC 51 as NSHP capture 272a with a confidence level of 98% 272b as shown by output 270 in this illustrative example. In FIG. 6B, the two EGM signal segments 256a and 256b received as first and second unknown signal inputs 252a and 252b, respectively, are classified as VMO capture 274a with a confidence level 274b of 92% as indicated by output 270. In the illustrative example of FIGS. 6A and 6B, the PCC classifier 51 has been trained using two channels of unknown cardiac electrical signal inputs sensed following the same VCS pacing pulse using two different sensing electrode vectors for classifying the VCS pacing pulse, e.g., as one of SHP capture, NSHP capture, VMO capture or LOC. The PCC output 270 may be used by external device processor 52 to generate data, information or other output according to any of the examples described herein, e.g., for generating a display of an annotated cardiac electrical signal with VCS pacing markers and capture type labels, determining and reporting capture threshold(s), and/or determining and recommending a VCS pacing pulse output setting.

FIGS. 6A and 6B represent one example of PCC 51 trained to classify VCS pacing pulses using two unknown cardiac electrical signal inputs. The two inputs may or may not be prepended or appended with other data, e.g., a value or values indicating the pacing pulse voltage amplitude and/or pacing pulse width of the associated VCS pacing pulse. As described above in conjunction with FIG. 4, PCC 51 may be trained to classify VCS pacing pulses using at least one unknown cardiac electrical signal input and one or more template beat inputs.

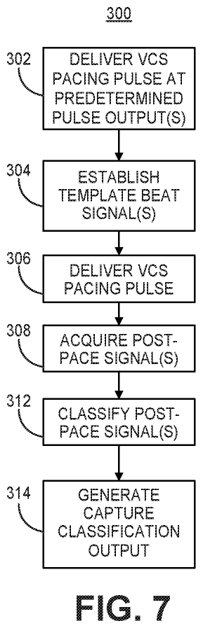

FIG. 7 is a flow chart 300 of a method that may be performed for classifying VCS pacing pulses by a medical device system according to another example using template beat inputs. In this example, one or more template beats are established by external device processor 52 or IMD control circuit 80 for use in providing input to PCC 51. At block 302 VCS pacing is delivered, e.g., by therapy delivery circuit 84 of IMD 14 or by an external pulse generator. One or more VCS pacing pulses may be delivered at a selected pacing pulse output. External device processor 52 or IMD control circuit 80 may receive a cardiac electrical signal sensed over at least the predetermined post-pace time interval following at least one VCS pacing pulse delivered at the selected pacing pulse output.

At block 304, a template beat signal may be established by the medical device system processing circuitry, e.g., external device processor 52 or IMD control circuit 80. The template beat signal may be sampled starting from one or more sample points prior to the VCS pacing pulse and during the post-pace time interval following a single paced beat in some examples. For example, a series of 2 to 10 VCS pacing pulses may be delivered at a selected pacing pulse output and the nth beat, e.g., the 3^{rd}, 5^{th} or other preselected nth paced beat, may be used for establishing the template beat signal. In other examples, the cardiac signal is obtained during at least the post-pace time interval following each of two or more, e.g., two to twelve or three to six VCS paced beats, where each VCS pacing pulse is delivered at the same pacing pulse output. The multiple cardiac signal segments may be ensemble averaged by processor 52 or control circuit 80 to obtain the template beat signal for the selected pacing pulse output. In some examples, when multiple cardiac signal segments are used to establish the template beat signal, each signal segment may be compared to at least one other signal segment to determine a cross-correlation between the signal segments. Any signal segment having a low cross-correlation with the other signal segments may be discarded.

In some examples, a single template beat signal is established at block 304 for one selected pacing pulse output, e.g., one pacing pulse amplitude that may be a highest pulse amplitude used during pacing capture threshold searches performed by IMD 14. In other examples, multiple template beat signals are established for each one of multiple selected pacing pulse outputs, e.g., two or more pacing pulse amplitudes with the same pulse width, two or more pulse widths with the same pulse amplitude, or multiple different combinations of pulse amplitude and pulse width. As such, the control circuit 80 of IMD 14 may be configured to control therapy delivery circuit 84 to deliver a predetermined number (e.g., 1 to 10 or 3 to 8) of VCS pacing pulses at each selected pacing pulse output at block 302. A template beat signal may be established using a cardiac electrical signal sensed during at least the post-pace time interval following at least one VCS pacing pulse delivered at each pacing pulse output.

The template beat signal may be sensed from the same sensing electrode vector as the sensing electrode vector used to obtain the unknown signal input to be classified. For example, the template beat signal may be established using an FF EGM signal sensed between the VCS lead tip electrode 32 or the VCS ring electrode 34 and the IMD housing. The unknown signal input (or at least one unknown signal input when multiple unknown signal inputs are received by PCC 51) may be sensed using the same FF EGM signal sensing electrode vector. In other examples, the template beat signal may be sensed using a different EGM or ECG sensing electrode vector than the sensing electrode vector used for obtaining the unknown signal input.

It is to be understood that the template beat signals established at block 304 may be of unknown capture type. The template beat signals provided to PCC 51 at different selected pacing pulse outputs provide PCC 51 with information about how much the unknown signal input to be classified differs from post-pace signal segments acquired during different VCS pacing pulse amplitudes (or pulse widths). The pacing pulse amplitude (or pulse width) may be known to PCC 51 as prepended or appended data in some examples.

At block 306, a VCS pacing pulse that is to be classified by PCC 51 is delivered. The VCS pacing pulse may be delivered at a currently programmed pacing pulse output used for delivering VCS pacing therapy. At other times, the VCS pacing pulse may be delivered at a test pacing pulse output as part of a capture threshold search. The VCS pacing pulse may be delivered by therapy delivery circuit 84 of IMD 14 and a post-pace signal may be sensed by the IMD sensing circuit 86 at block 308. Control circuit 80 may obtain the unknown input signal as an EGM signal segment that is sensed during the post-pace time interval and store the EGM signal segment in memory 82 for transmitting to external device 50. In other examples, control circuit 80 may transmit an EGM signal received from sensing circuit 86 in real time or after storing in memory 82, and external device processor 52 may extract the desired EGM signal segment, including at least a post-pace time interval, from the transmitted EGM signal. As described above, the EGM signal segment may include one or more pre-pace sample points for providing a reference time point for aligning multiple input signals and for baseline zeroing.

At block 312, the unknown signal input(s) obtained following the VCS pacing pulse to be classified and the previously established template beat input(s) are passed to PCC 51. PCC 51 is previously trained to classify the unknown signal input(s) and associated VCS pacing pulse according to capture type using the number and types of input received. At block 314, PCC 51 generates a capture classification output, which may include a level of confidence of the capture classification, determined by applying the machine learning model to the unknown signal input(s) and the template beat input(s). As described above, the capture classification may be one of SHP capture, NSHP capture, VMO capture or LOC or any other capture or beat types that PCC 51 is trained to classify. In some examples, the PCC output includes a level of confidence associated with each possible capture type classification, with the highest level of confidence being the determined capture type classification of the delivered VCS pacing pulse. The PCC output may be stored in external device memory 53 for use by external device processor 52 (or transmitted and stored in IMD memory 82 for use by control circuit 80), e.g., according to any of the example monitoring, reporting and/or pacing therapy control functions described herein.

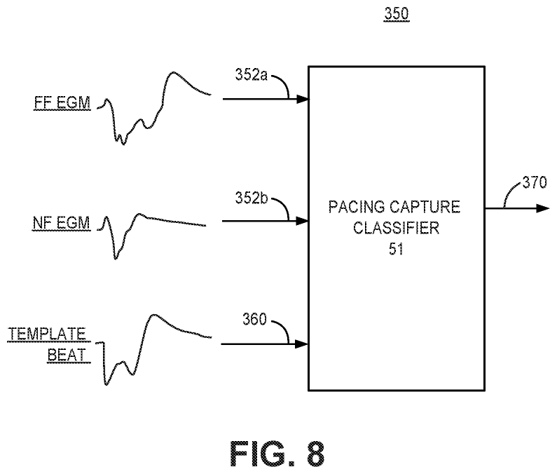

FIG. 8 is a conceptual diagram 350 of the processing circuit PCC 51 configured to analyze cardiac signals for determining a pacing capture classification according to another example. In this example, PCC 51 is trained to classify a delivered VCS pacing pulse based on an FF EGM signal segment received as a first unknown signal input 352a, an NF EGM signal segment received as a second unknown signal input 352b, and a template beat input 360. Each of the first and second unknown signal inputs 352a and 352b are obtained from a respective FF EGM signal and NF EGM signal sensed during at least the post-pace time interval following delivery of the VCS pacing pulse that is being classified. PCC 51 is trained to process the three inputs by applying the machine learning model to the three inputs to determine a capture type classification output 370 of the VCS pacing pulse, e.g., as generally described above in conjunction with FIG. 7. Each of the signal inputs 352a, 352b and 360 may be time aligned starting with the first sample point of the respective signal input, which may be a pre-pace sample point. The first sample point may be zeroed for each signal input to zero the baseline of each signal 352a, 352b and 360.

The template beat input 360 can be established as described above in conjunction with FIG. 7 from a cardiac electrical signal, e.g., one of the FF EGM signal or the NF EGM signal, sensed during one or more post-pace time intervals following a VCS pacing pulse delivered at a predetermined, selected pacing pulse output. The template beat input 360 may be established for a VCS pacing pulse having a relatively high pacing pulse amplitude, e.g., 4 volts or higher or 5 volts or higher, with a pulse width of at least 0.25 to 0.5 ms, as examples. The template beat may be established for a cardiac signal sensed during VCS pacing at a relatively high pacing pulse output so that the template beat corresponds to the QRS waveform when at least a portion of the VCS is expected to be captured. The actual capture type of the template beat, however, may be unknown to PCC 51.

In other examples, PCC 51 may be trained to determine a capture type classification output 370 based on applying the machine learning model to the template beat input 360 and only one unknown signal input, e.g., the FF EGM signal input 352a or the NF EGM signal input 352b or one ECG signal input. Multiple unknown signal inputs are not necessarily required for classification of a VCS pacing pulse and the subsequent cardiac electrical signal waveform. In one example, PCC 51 is trained to classify an unknown signal input 352a received as an FF EGM signal input obtained following a VCS pacing pulse of unknown capture type and a template beat input 360 established from the FF EGM signal sensed following at least one VCS pacing pulse delivered using a 5.0 volt pulse amplitude and 0.2 to 0.5 ms pulse width (or any other selected pacing pulse output).

PCC 51 may additionally receive data input indicating the pacing pulse voltage amplitude of the VCS pacing pulse that is being classified and/or the pacing pulse voltage amplitude of the template beat input. The pacing voltage amplitude may be prepended or appended to at least one of the unknown signal inputs 352a or 352b and/or the template beat input 360 or provided as a separate input signal in various examples. Additionally or alternatively, PCC 51 may receive a data input indicating a signal feature of the unknown input signal 352a and/or 352b and/or a feature of the template beat input 360, as generally described above in conjunction with FIG. 4.

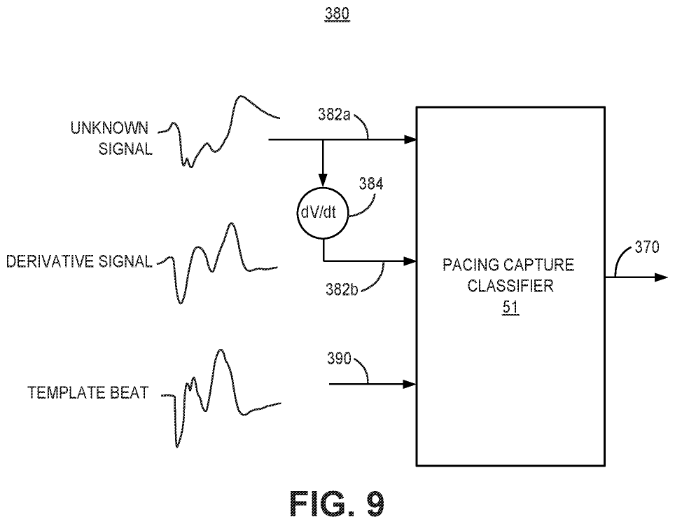

FIG. 9 is a diagram 380 of PCC 51 according to another example. In this example, PCC 51 may be trained to provide a capture type classification output 370 based on an unknown signal input 382a, a derivative signal input 382b, and a template beat input 390. The unknown signal input 382a may be an FF EGM signal, NF EGM signal, or an ECG in various examples. In one example, the unknown signal input 382a may be obtained from the FF EGM signal sensed during at least the post-pace time interval using a tip-to-can sensing electrode vector, e.g., from the VCS lead tip electrode 32 to the IMD housing 15 (shown in FIGS. 1A-1C, for example). The derivative signal input 382 may be determined by control circuit 80 or external device processor 52 as a derivative of the unknown signal input 382a. In some examples, the derivative signal input 382 is a first derivative signal, but in other examples the derivative signal input 382 may be determined as a higher order derivative, e.g., second, third or higher derivative. Processing circuitry included in control circuit 80 may include a differentiator 384, for example, for obtaining the derivative signal from the unknown signal input 382a. Differentiator 384 may be implemented in hardware, firmware or software in control circuit 80 or external device processor 52 for determining the first order derivative of the unknown signal input 382a to be provided as the derivative signal input 382b. It is contemplated that in some examples PCC 51 is trained to classify VCS pacing pulse capture type in response to receiving only the derivative signal input 382 or the derivative signal input 382 and any combination of one or more unknown signal inputs, other derivative signal inputs, and/or template beat inputs.

As such, at block 308 of FIG. 7, control circuit 80 or external device processor 52 may operate to acquire the post-pace signals for providing input to PCC classifier 51 by obtaining the unknown input signal 382a sensed over at least the post-pace time interval following the VCS pacing pulse that is being classified and determining a derivative of the unknown input signal 382a. The template beat input 390 may be previously established as described above in conjunction with FIG. 7 such that control circuit 80 or external device processor 52 is configured to provide each of the three inputs 382a, 382b and 390 to PCC 51 for producing the capture type classification output 370.

While not shown in the example of FIG. 9, it is contemplated that in addition to or alternatively to the derivative signal 382b determined from the unknown signal input 382a, control circuit 80 or external device processor 52 may be configured to determine a derivative of the template beat input 390. In some examples, the derivative of the established template beat may be provided instead of the template beat signal itself as the input 390 to PCC 51. In other examples, both of the template beat signal and a derivative of the template beat signal may be provided as inputs to PCC 51.

Furthermore, while one derivative signal input 382b is shown in FIG. 9, it is to be understood that a first order derivative signal and a second order or other higher order derivative signal determined from the unknown input signal 382a may be provided as input to PCC 51. Additionally or alternatively, a first order derivative signal and a second order or other higher order derivative signal determined from the template beat signal 390 may be provided as input to PCC 51. Thus, PCC 51 may be trained to receive one or more derivative signals determined from the unknown input signal 382a and/or one or more derivative signals determined from the template beat input 390 as additional input signal(s). The derivative signals may be determined as nth order difference signals from the respective unknown input signal or template beat signal.

It is to be understood that additional data input such as the pacing voltage amplitude, pacing pulse width, or other VCS pacing pulse information and/or one or more signal features of the unknown signal input 382a, derivative signal input 382b, template beat input 390 and/or template beat derivative input (if included), may be determined by control circuit 80 or external device processor 52 and provided as input to PCC 51. Additional data may be provided as a separate input and/or prepended or appended to one of the signal inputs 382a, 382b or 390. A determined signal feature may be a maximum peak amplitude, peak-to-peak amplitude, maximum (positive) slope, minimum (negative) slope, signal width, signal area, time between the maximum slope and the minimum slope, an activation time from a delivered VCS pacing pulse to a maximum peak or another fiducial point of the signal segment or any other signal feature listed herein.

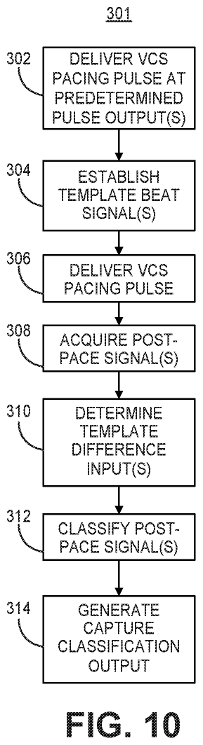

FIG. 10 is a flow chart 301 of a method for determining a VCS pacing capture type according to another example. Identically numbered blocks shown in FIG. 10 correspond to like-numbered blocks shown in FIG. 7 and described above. In the example of FIG. 10. however, after acquiring the post-pace signal(s) for passing at least one unknown signal input to PCC 51 at block 308, control circuit 80 or external device processor 52 may determine a template difference input at block 310 as the difference between an unknown signal input and an established template beat signal. At block 312, PCC 51 may receive at least one unknown input signal, e.g., at least one post-pace FF EGM signal segment, NF EGM signal segment and/or ECG signal segment or any combination thereof, to be classified (along with the delivered VCS pacing pulse) and at least one template difference input determined at block 310.

In various examples, a template difference input may be determined between each of multiple template beat signals established at block 304 and an unknown input signal sensed from the same sensing electrode vector. Multiple template difference inputs may be received by PCC 51 at block 312 for classifying capture type of the VCS pacing pulse delivered at block 306. When PCC 51 is trained using multiple unknown signal inputs, each obtained from different sensing electrode vectors, PCC 51 may additionally receive multiple template difference inputs corresponding to the difference between an established template beat signal at a predetermined pacing pulse output and a post-pace unknown signal input obtained at block 308 and sensed from the same sensing electrode vector as the established template beat signal. PCC 51 may be trained to classify the VCS pacing pulse capture type using at least one unknown input signal and at least one template difference signal. The template beat signal used to determine the difference signal may additionally be provided as an input signal to PCC 51 in some examples.

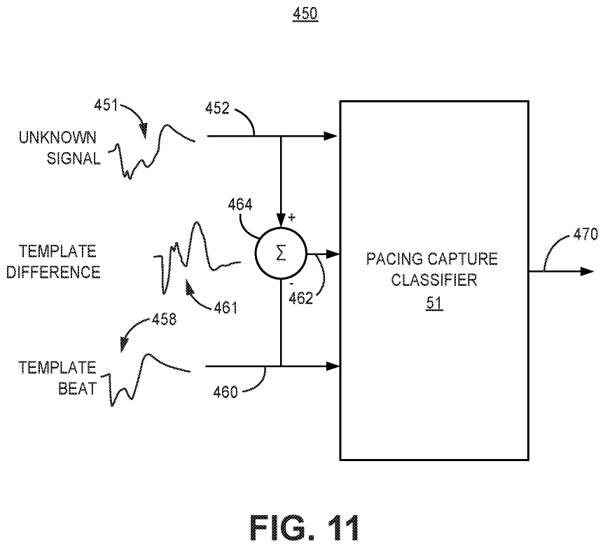

FIG. 11 is a conceptual diagram 450 of PCC 51 according to yet another example. In this example, PCC 51 receives at least one unknown input signal 452 acquired from a cardiac electrical signal (shown as unknown signal segment 451) sensed during at least the post-pace time interval following delivery of the VCS pacing pulse to be classified. A template beat signal 458 is previously established by control circuit 80 or external device processor 52 as described above and may be provided as a template beat input 460. The template beat signal 458 may be determined from the post-pace signal sensed from the same sensing electrode vector as the unknown signal segment 451 following a VCS pacing pulse delivered at a predetermined pacing pulse output, e.g., pulse amplitude of 5.0 volts or higher or another relatively high pacing pulse output expected to typically capture cardiac tissue, which may include at least a portion of the VCS. In other examples, the template beat signal 458 may correspond to a post-pace signal obtained following a VCS pacing pulse delivered at any predetermined or pre-selected pacing pulse output. The unknown signal segment 451 and the template beat signal 458 may be time aligned starting with the first sample point of the respective signal segments, which may be a pre-pace sample point. The baseline of the unknown signal segment 451 and template beat signal 458 may be zeroed by zeroing the first sample point of each signal. The time aligned and zeroed signals 451 and 460 may be provided to PCC 51 as multi-channel data point inputs 452 and 460.

In addition, in the example of FIG. 11, control circuit 80 or external device processor 52 can be configured to determine the template difference signal 461 by subtracting the template beat signal 458 from the unknown signal segment 451 (or vice versa) to obtain the template difference signal 461.

Control circuit 80 or external device processor 52 may include a summation (or difference) circuit that may be implemented as hardware, firmware and/or software configured to determine the difference between each sample point of the time-aligned and zeroed unknown signal segment 451 and the template beat signal 458. In the example of FIG. 11, control circuit 80 or external device processor 52 includes a summation circuit 464 configured to perform a summation of the positive input of the unknown signal input 452 and the negative input of the template beat signal 458 (or vice versa) for providing a template difference input 462 to PCC 51.

PCC 51 is trained to output a capture type classification 470 based on applying a machine learning model to at least the unknown signal input 452 and the template difference input 462. In some examples, as shown in FIG. 11, PCC 51 may additionally receive the template beat input 460 and is trained to output the capture type classification 470 based on the three inputs of the unknown signal input 452, template beat input 460 and the template difference input 462. However, the template beat input 460 may be optional and omitted in some examples when the template difference input 462 is provided to PCC 51.

As generally described above, control circuit 80 or external device processor 52 may be configured to provide PCC 51 with one or more additional data inputs, such as a signal feature determined from one or more of the unknown signal segment 451, template beat signal 458 and/or template difference signal 461, and/or the pacing pulse output of the delivered VCS pacing pulse to be classified or the pacing pulse output associated with the template beat signal. PCC 51 may be trained to provide the capture type classification output 470 based on the input signals 452, 460 and 462 and one or more data inputs providing information about the delivered VCS pacing pulse to be classified and/or a specific signal feature of the unknown signal segment 451, template beat signal 458 and/or template difference signal 461. The additional data inputs may be received by PCC 51 as prepended or appended data of one of the inputs 452, 460 or 462 or as one or more separate input signals.

In an illustrative example, PCC 51 may receive a feature of the template beat signal 458 and may or may not receive the template beat input 460 when a template beat feature is received. For example, control circuit 80 or external device processor 52 may determine a signal feature of template beat signal 458, such a maximum peak amplitude, a signal width, a maximum slope, and/or a time from the VCS pacing pulse to the maximum peak, maximum slope, or any other signal feature or combination of features of the template beat signal 458. PCC 51 may receive the determined template beat signal feature(s) as input along with at least the unknown signal input 452 and the template difference input 462 for determining a capture type classification output 470 in some examples.

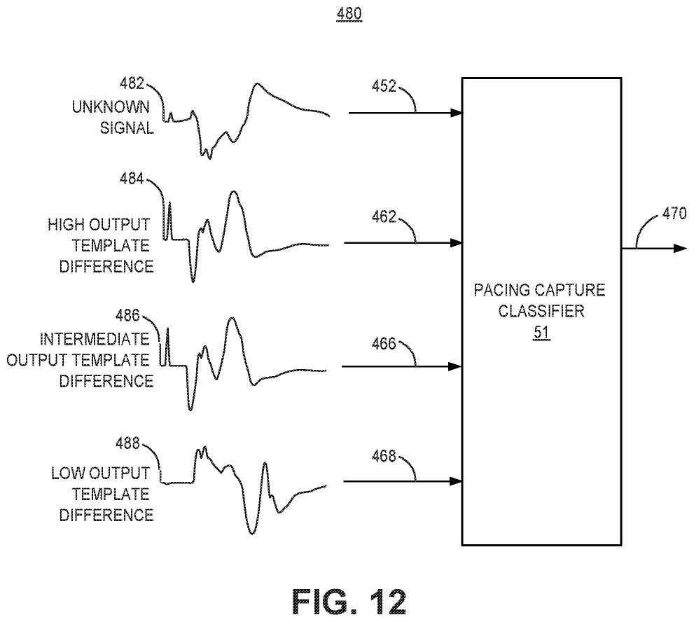

FIG. 12 is a conceptual diagram 480 of PCC 51 according to another example. In this example, PCC 51 is trained to classify an unknown signal input 452 (and the corresponding delivered VCS pacing pulse) based on the unknown signal input 452 and multiple template difference inputs. In the example shown, PCC 51 receives three template difference inputs 462, 466 and 468. Each template difference input 462, 466 and 468 may be determined by control circuit 80 or external device processor 52 as the difference between the unknown signal input 452 and a template beat signal established during VCS pacing at a respective, predetermined pacing pulse output. The four signal inputs, the unknown signal input to be classified and the three template difference inputs, may be combined into a 4-channel data point, represented by inputs 452, 462, 466 and 468, received by PCC 51.

In the example shown, the processing circuitry of control circuit 80 or external device processor 52 may determine three different template beat signals corresponding to three pre-determined different VCS pacing pulse outputs, e.g., a relatively high output, a relatively low output and an intermediate output that may be mid-range between the high and low outputs. In other examples two template beat signals may be established for a relatively high output and a relatively lower output. In still other examples, more than three template beat signals may be established including a high, low and two or more intermediate output template beat signals. For each template beat signal, the processing circuitry may determine a template difference signal as the difference between the unknown signal acquired during the post-pace time interval of the VCS pacing pulse to be classified and each of the established template beat signals. As described above, the unknown signal and the template beat signals may be time aligned starting with the first sample point of the respective signal segment (which may be a pre-pace sample point), and the baseline of each of the signals may be zeroed by zeroing the first sample point of each signal prior to determining the respective template difference signals.

Each template difference signal can be provided as an input signal to PCC 51. As shown in FIG. 12, a high output template difference input 462, an intermediate output template difference input 466 and a low output template difference input 468 may be provided as input to PCC 51. In one example, the high output template beat signal is obtained during VCS pacing delivered at a highest test pacing pulse output, e.g., between 5 and 8 volts pulse amplitude, used during a VCS pacing capture threshold test by IMD 14. The low output template beat signal is obtained during VCS pacing delivered at a lowest test pacing pulse output used during a VCS pacing capture threshold test, e.g., between 0.5 and 1.5 volts pulse amplitude. The intermediate template beat signal may be determined at a pacing pulse output midway between the high and low outputs.

PCC 51 is trained to determine a capture type classification output 470 based on the four inputs 452, 462, 466 and 468 in this example. In some examples, one, two or all three of the template beat signals used to determine the template difference inputs 462, 466 and 468 may also be provided as inputs to PCC 51 in addition to the template difference inputs. When PCC 51 is trained to produce the capture type classification output 470 using at least one template difference input, any differences between the waveform morphology of the unknown input signal to be classified and the established template beat signal may be learned more quickly or efficiently during training of PCC 51 according to a machine learning algorithm. Differences between the unknown signal input 452 and the any of the template beat signals established at multiple different pacing pulse amplitudes are directly provided to PCC 51 as the template difference input(s).

As described above, any of the signal inputs 452, 462, 466 and/or 468 may be prepended or appended with additional data such as an associated pacing voltage amplitude, pacing pulse width, a specific signal feature and/or patient information (e.g., age, sex, etc.) as described in any of the examples given above. In the example shown, each signal input 452, 462, 466 and 468 is prepended with a VCS pacing pulse voltage amplitude 482, 484, 486 and 488, respectively, encoded in the respective signal input 452, 462, 466 and 468. In other examples, PCC 51 may receive additional pacing pulse related data and/or signal feature related data as a separate input, not prepended or appended to a signal input. Furthermore, it is to be understood that any of the examples of one or more unknown signal inputs, one or more unknown signal derivative inputs, one or more template beat inputs, one or more template difference inputs, one or more VCS pacing pulse related data inputs and/or one or more signal feature data inputs may be received by PCC 51 in any selected combination during a machine learning training session and subsequently during PCC operation for classifying VCS pacing pulses. The conceptual diagrams of PCC 51 presented in FIGS. 4, 6A, 6B, 8, 9, 11 and 12 are illustrative in nature and not intended to be limiting of specific types and combinations of inputs that PCC 51 may receive during training and during subsequent VCS pacing pulse classification operations. The various input signals or channels shown or described herein may be received by PCC 51 in different combinations than the particular combinations shown in the accompanying figures and described herein and/or one or more input signals shown in the illustrative examples may be omitted.

Furthermore, it is to be understood that various operations may be performed on one or more unknown input signals and/or one or more template beat signals by the medical device system processing circuitry for obtaining input signals provided to PCC 51, which may include additions, subtractions, determining integrals, and/or determining derivatives of a cardiac electrical signal segment as examples. In some cases, two or more operations may be performed. For example, a template difference signal may be determined between an unknown signal input and an established template beat signal and a derivative signal may be determined from the template difference signal for providing a template difference derivative input to PCC 51.

FIG. 13 is a flow chart 400 of a method for generating VCS pacing pulse capture type classifications during a pacing capture threshold test according to some examples. At block 402, IMD 14 may perform a capture threshold test. Control circuit 80 may control therapy delivery circuit 84 to deliver VCS pacing pulses to a His-Purkinje pacing site using a selected pacing electrode vector, e.g., according to any of the examples of FIGS. 1A-2B. Therapy delivery circuit 84 delivers the VCS pacing pulses at multiple pacing pulse output settings. For the sake of convenience, the capture threshold search is described as being performed by varying the pacing pulse amplitude using a fixed pacing pulse width. It is recognized however, that a capture threshold search may be performed by varying the pacing pulse width using a fixed pacing pulse amplitude or varying both the amplitude and the pulse width. In an example, one or more VCS pacing pulses are delivered at each of multiple pacing pulse amplitudes starting from a relatively high pulse amplitude, e.g., 5.0 volts or higher, and decreasing to a relatively low pulse amplitude, e.g., 1.0 volt or lower. The VCS pacing pulse amplitude may be decreased in 0.25, 0.5 or 1 volt steps as examples. In other instances, the pacing pulse amplitude may start at a low amplitude and be increased or be randomly varied.

At block 404, the processing circuit in which PCC 51 is implemented receives the capture threshold test data. For the sake of convenience, external device processor 52 is described as receiving the capture threshold test data. In other examples, however, control circuit 80 may be configured to perform the analysis of the capture threshold test data according to the process of FIG. 13. During the capture threshold search performed by IMD 14, sensing circuit 86 passes one or more EGM signals to control circuit 80 that can be transmitted to processor 52 via telemetry. Processor 52 may receive the capture threshold test data at block 404 by receiving at least one EGM signal, e.g., an FF EGM signal, which may be transmitted in real time by IMD 14, along with associated VCS pacing pulse timing markers and pacing pulse output data. Alternatively, segments of the FF EGM signal representative of the FF EGM signal during at least a post-pace time interval following VCS pacing pulses delivered at each pacing voltage amplitude applied during the capture threshold test may be stored in IMD memory 82 and subsequently transmitted to external device 50.

External device processor extracts the inputs needed for PCC 51 from the received capture threshold test data. For example, at block 406, external device processor 52 may establish one or more template beat signals from the EGM signal(s) received from IMD 14 for one or more pre-selected pacing pulse voltage amplitudes that are applied during the capture threshold test. Template beat signals may be established as generally described above in conjunction with FIG. 12.

At block 408, processor 52 may extract the first unknown signal input to be classified by PCC 51. The unknown signal input can be extracted from the EGM signal received from IMD 14 during at least a post-pace time interval following a capture test pacing pulse. At block 410, processor 52 may determine the template difference input for each template beat signal determined at block 406. Processor 52 may determine the difference between each time aligned sample point of the unknown signal input and a template beat signal to determine a template difference input. As described above, multiple template difference inputs may be determined and provided in a multi-channel input to PCC 51. The unknown signal input and the determined template difference input(s) are provided as input to PCC 51 at block 412.

External device processor 52 may receive or determine any other PCC input signals required for capture type classification, in accordance with the input signals that PCC 51 was previously trained on. PCC input signals may be received or determined by processor 52 for providing as input to PCC 51 according to any of the examples described above.

At block 412, processor 52 determines a capture type classification by applying the machine learning model of PCC 51 to the unknown input signal and any other input signals. PCC 51 may produce the capture type classification output of the unknown signal input (and associated VCS capture test pacing pulse). The capture type classification output by PCC 51 may include the level of confidence of the capture type classification. In some examples, PCC 51 may output the level of confidence of each possible type of capture classification that PCC 51 is trained to identify.

At block 414, external device processor 52 receives the PCC output and may store the VCS pacing pulse capture type classification and associated pacing pulse amplitude for use in generating an annotated display of the capture threshold test results. If another VCS pacing pulse remains to be classified from the capture threshold test, as determined by processor 52 at block 416, processor 52 may return to block 408 to obtain the required PCC input signals for determining a capture type classification of the next VCS pacing pulse by applying the machine learning model of PCC 51 until all VCS pacing pulses of the capture threshold test have been classified.

At block 418, external processor 52 may use the PCC output classifications and stored EGM signal data and/or VCS pacing pulse output data to generate a representation of the capture threshold test results. External processor 52 may display at least one cardiac electrical signal and/or a VCS pacing marker channel to present to a clinician or other user the results of the capture threshold test. The EGM signal received from IMD 14 used for obtaining the inputs to PCC 51 may be displayed and annotated with the capture type classifications, along with the VCS pacing markers labeled according to pacing pulse output (e.g., pulse amplitude and/or pulse width). Additionally or alternatively, any other ECG and/or EGM signal sensed during the capture threshold test and received by processor 52 may be annotated with the capture type classifications or displayed in time alignment with the capture type classifications.

In some examples, all capture threshold test pulses are classified by processor 52 and the capture test results are generated for display at block 418 after capture type classifications of all capture threshold test VCS pacing pulses is complete. In other examples, the annotated EGM and/or ECG signals may be displayed as capture test classifications are determined from the output of PCC 51. In various examples, display unit 54 may display the results of the capture threshold test in a rolling time-based signal, in a table and/or in a graph, e.g., listing and/or plotting each VCS pacing pulse amplitude and the associated capture type classification.

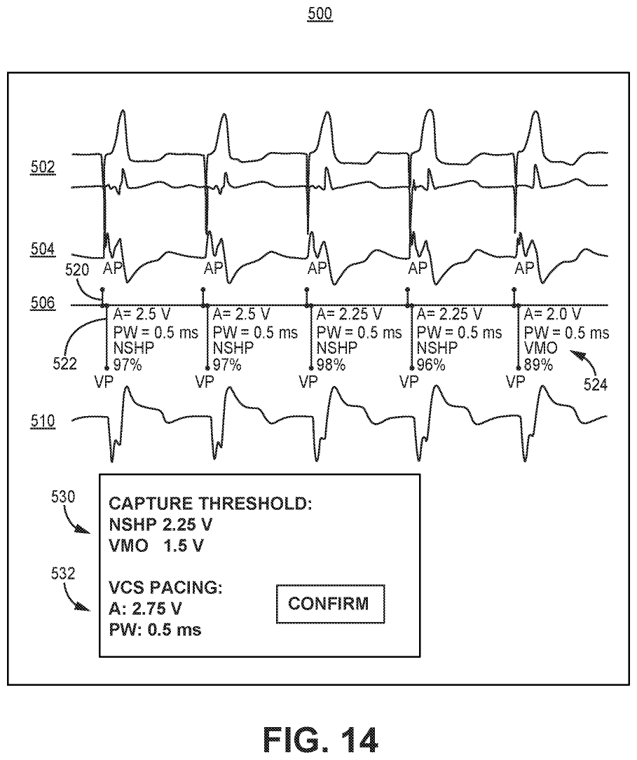

FIG. 14 is a conceptual diagram 500 of cardiac electrical signals and a marker channel that may be generated by external device processor 52 for display by display unit 54 using the capture type classifications output by PCC 51 according to one example. Diagram 500 may represent a GUI that may be displayed by display unit 54. PCC 51 may be trained to output capture type classification of VCS pacing pulses according to any of the examples described above. Processor 52 and display unit 54 may receive one or more ECG signals 502, e.g., via interface 55 shown in FIG. 1A, for displaying ECG signals that are sensed simultaneously with EGM signals sensed by IMD 14. An atrial EGM signal 504 and a ventricular EGM signal 510 may be sensed by IMD sensing circuit 84 and transmitted by telemetry from IMD 14 to external device 50. The atrial EGM signal 504 may be sensed by the RA lead electrodes 20 and 22. The ventricular EGM signal 510 may be an FF EGM signal, e.g., a tip to can or ring to can signal, as shown in this example, or a NF EGM signal, e.g., a tip to ring signal. The ventricular EGM signal 510 may be the signal sensed by sensing circuit 86 using a sensing electrode vector that is used to obtain the unknown signal input and one or more template beat signals for obtaining input signals received by PCC 51. However, external device 50 may receive and display cardiac electrical signals, including ECG and/or EGM signals, in addition to or different than the signal(s) used by processing circuitry of external device 50 or IMD 14 for obtaining signal inputs received by PCC 51.

The marker channel 506 includes atrial pacing pulse (AP) markers 520 and VCS pacing pulse (VP) markers 522 to mark the relative timing of respective atrial pacing pulses and VCS pacing pulses delivered by IMD 14. Each VCS pacing pulse may be delivered at a shortened AV interval from the atrial pacing pulse during the capture threshold test (to pace the ventricles earlier than any intrinsically conducted depolarizations). Each VCS pacing pulse marker 522 may be annotated with the pacing pulse amplitude (A), pulse width (PW), capture type classification output by PCC 51 and the level of confidence expressed as a percentage if available. External device processor 52 may generate the capture type annotations 524 as an output in response to the capture type classifications made by PCC 51 (and corresponding levels of confidence) for display to a user, which may be in combination with at least one time aligned cardiac electrical signal.

In some examples, external device processor 52 may determine a VCS pacing capture threshold based on the PCC output. For example, processor 52 may determine a lowest VCS pacing pulse amplitude at which each type of capture type classification output by PCC 51 occurred during a capture threshold test. In the example shown, NSHP capture is determined for pacing pulse amplitudes at 2.5 and 2.25 volts according to the annotations 524. VMO capture is determined when the pacing pulse amplitude is decreased to 2.0 volts as shown by annotations 524. The lowest pulse amplitude at which VMO capture is determined is not displayed in the diagram of FIG. 14. However, based on the PCC output during a capture threshold test, external device processor 52 may determine a SHP capture threshold, a NSHP capture threshold and/or a VMO capture threshold. In the hypothetical example shown, processor 52 may determine a NSHP capture threshold as 2.25 V and a VMO capture threshold of 1.5 volts (not shown by marker channel 506 in FIG. 14). The determined capture thresholds may be reported in the capture threshold display window 530.

The determined capture thresholds may be the lowest VCS pacing pulse amplitude at which at least X out of Y capture classifications are the same and the level of confidence is at least a predetermined threshold percentage. For example, each test pacing output may be delivered for at least 3 VCS pacing pulses, 5 VCS pacing pulses or another selected number of test pacing pulses during the capture threshold test. When at least 2 out 3 or 3 out of 5 or other selected portion of the VCS pacing pulses delivered at a test output are classified the same, and each of the same classifications have a level of confidence of at least 70% (or any another predetermined threshold percentage), processor 52 may determine same capture type classifications as the capture type classification of the test pacing pulse output. The lowest test output resulting in that same capture type classification may be determined as the capture threshold for that capture type.

Depending on the progression of capture type as the pacing pulse output is increased or decreased, one or more capture types may not be determined by processor 52 based on the output of PCC 51. For example, in some instances NSHP capture may occur at the highest pacing pulse output and capture of the His-Purkinje system may be lost as pacing pulse output is decreased before VMO capture is lost. In this case, PCC 41 may output NSHP capture classifications, VMO capture classifications, and LOC classifications but not SHP classifications. Processor 52 may determine a NSHP capture threshold and a VMO capture threshold, as shown in the example of FIG. 14. In other examples, however, ventricular myocardial capture may be lost before selective capture of the His-Purkinje system. In this case, processor 52 may determine a NSHP capture threshold and a SHP capture threshold and a VMO capture threshold may be indeterminable because capture of both the His-Purkinje system and ventricular myocardium occur together at or above the NSHP capture threshold and only SHP capture may occur below the NSHP capture threshold, with LOC occurring at pacing pulse amplitudes less than the SHP capture threshold.

In still other examples, SHP capture may occur at all VCS pacing pulse outputs until LOC is determined such that only a SHP capture threshold is determined. In still other examples, NSHP capture may occur at all VCS pacing pulse outputs until LOC is determined such that only NSHP capture threshold is determined. Accordingly, the capture thresholds of different capture classification types that are determinable and indeterminable may vary in different patients and/or over time.

External device processor 52 may be configured to output a recommended operating VCS pacing output setting based on the capture type classifications output by PCC 51, which may be displayed in a recommended settings window 532. Processor 52 may determine a recommended operating VCS pacing output setting as a pacing pulse amplitude and/or width that is a safety margin, e.g., 0.25 to 2.0 volts greater than a determined SHP or NSHP capture threshold. In the illustrative example of FIG. 14, processor 52 may generate a recommended operating VCS pacing amplitude setting of 2.75 volts, which is a 0.5 volt safety margin greater than the NSHP capture threshold of 2.25 volts. In some examples, a user may review and accept or confirm a recommended operating VCS pacing output setting. For example, a user may click the “confirm” button of the recommended settings window 532 to program the recommended operating settings in IMD 14, which may be transmitted by external device telemetry unit 58 for receipt by IMD 14. In other instances, the user may select and program an operating VCS pacing pulse amplitude and/or pulse width based on the visual representations of the cardiac electrical signal(s) and the capture type classifications presented in a GUI by display unit 54. IMD therapy delivery circuit 84 may be subsequently controlled to deliver VCS pacing pulses according to the programmed operating VCS pacing pulse output.

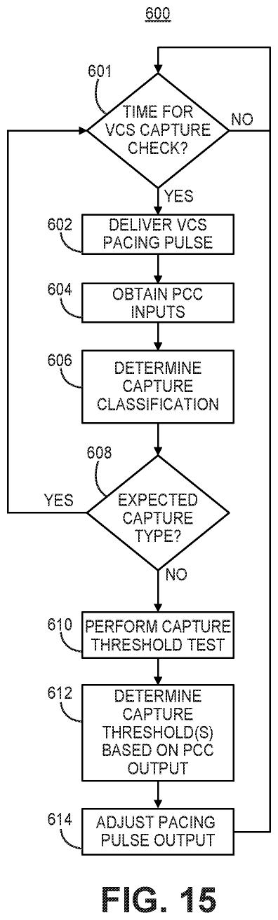

FIG. 15 is a flow chart 600 of a VCS capture monitoring method that may be performed by processing circuitry of a medical device system according to some examples. For the sake of case, FIG. 15 is described as being performed by control circuit 80 of IMD 14, e.g., in conjunction with therapy delivery circuit 84, sensing circuit 86 and memory 82 and any other circuits and components of IMD 14 as needed. The machine learning model of PCC 51 may be implemented in control circuit 80 and may be previously trained using machine based learning algorithms for classifying an unknown signal input according to capture type using any of the example techniques described above. For example, training of PCC 51 may be completed by processing circuitry in external device 50 and after the machine learning model is fixed, PCC 51 may be programmed into IMD control circuit 80. In other examples, the process of flow chart 600 may be performed, all or in part, by an external computing system, such as external device 50 shown in FIG. 1A.

At block 601, control circuit 80 may determine if it is time to check for VCS capture. Control circuit 80 may be configured to check for VCS capture on a periodic or scheduled basis, which may be once per day, once per hour, once per minute or other scheduled frequency. In one example, control circuit 80 may be configured to perform the process of flow chart 600 once per day, e.g., at a scheduled time during nighttime hours, which may be between midnight and 4 a.m. as an example or at a time when a patient is expected to be resting. In other examples, control circuit 80 may be configured to check for VCS capture relatively more frequently, e.g., after every predetermined number of delivered VCS pacing pulses, e.g., after every 5 to 120 VCS pacing pulses. In some examples, control circuit 80 may be configured to determine VCS capture on a beat-by-beat basis although the processing power required to classify VCS capture type using PCC 51 on a beat by beat basis may unacceptably shorten the life of the IMD power source 98.

Additionally or alternatively, control circuit 80 may perform a VCS capture check on a triggered basis, e.g., in response to detecting a change in a lead impedance measurement, a pacing mode switch, or in response to detecting a change in the post-pace QRS morphology or any other detected triggering event that may indicate a possible change in VCS pacing pulse capture type. In some examples, control circuit 80 may determine that it is time to check for VCS capture based on determination of a change in a post-pace QRS signal morphology or signal feature (e.g., peak amplitude, peak slope, activation time, signal width or other signal feature) that may be monitored on a beat-by-beat or relatively more frequent basis than the VCS capture check is performed. Control circuit 80 may determine that it is time to perform a VCS capture check at block 601 in response to detecting a change in a post-pace EGM signal feature or QRS morphology compared to a previously determined post-pace EGM signal feature or QRS morphology or compared to a predetermined or previously established threshold value applied to a post-pace EGM signal feature.

Upon determining that it is time to check for VCS capture at block 601, control circuit 80 may wait for the next VCS pacing pulse delivered by therapy delivery circuit 84 at block 602. The VCS pacing pulse may be delivered at the current pacing pulse output settings, e.g., the currently programmed pulse amplitude and pulse width. In some examples, control circuit 80 may control therapy delivery circuit 84 to deliver the VCS pacing pulse at a shortened pacing interval, e.g., at a shortened AV delay or shortened ventricular pacing rate interval to avoid a fusion beat or an intrinsic ventricular depolarization prior to the delivered VCS pacing pulse.

At block 604, control circuit 80 obtains the input signals required for PCC 51. corresponding to the input signals used for training PCC 51. As described above, the PCC inputs include at least one unknown signal input obtained from a sensing electrode vector signal sensed during at least a post-pace time interval following the VCS pacing pulse delivered at block 602. Control circuit 80 obtains any additional input signals required which may include multiple unknown signal inputs obtained from EGM signals sensed during the post-pace time interval, one or more derivatives of the unknown signal input(s), one or more template beat inputs, and/or one or more template difference inputs. Furthermore, control circuit 80 may obtain additional input data relating to an associated VCS pacing pulse output and/or a specific signal feature determined from any of the input signals as described in foregoing examples herein.

PCC 51 receives the input signals and outputs a capture type classification based on the machine learning model of PCC 51 applied to the input signals at block 606. Control circuit 80 determines if the output capture type is the expected capture type at block 608. For example, control circuit 80 may determine if the output capture type is either of an SHP or NSHP capture type. In some examples, the expected output capture type may be selective or non-selective capture of a targeted portion of the His-Purkinje system, e.g., selective or non-selective LBB capture without RBB block. In some cases, the expected output capture type can be the capture type previously determined to occur when a VCS pacing pulse was delivered using the same pacing pulse output settings as the VCS pacing pulse delivered at block 602. When the capture classification is the expected capture type, control circuit 80 may return to block 601 to wait for the next VCS capture check. When the output capture type classification received from PCC 51 is not an expected capture type, e.g., not SHP or NSHP capture, LOC or VMO capture may be occurring. As such, control circuit 80 may advance to block 610 to perform a capture threshold test.

At block 610, control circuit 80 may perform a capture threshold test by controlling therapy delivery circuit 84 to deliver VCS pacing pulses at each of multiple different pacing pulse outputs (e.g., as generally described above in conjunction with FIG. 13). Control circuit 80 receives EGM signals from sensing circuit 86 and obtains the necessary input signals and data for PCC 51 after each VCS pacing pulse. Control circuit 80 may store the PCC output capture type classification associated with each VCS pacing pulse of the capture threshold test in memory 82. For example, therapy delivery circuit 84 may deliver VCS pacing pulses at each pacing voltage amplitude over a test range, e.g., over a range starting from 5 volts decreasing down to 1 volt or until LOC is the determined capture type output by PCC 51. Therapy delivery circuit 84 may deliver one or more VCS pacing pulses at each pacing pulse amplitude. For example, three to five VCS pacing pulses may be delivered at each pacing pulse amplitude. The capture type classification may be output by PCC 51 for each VCS pacing pulse and may include the level of confidence of the capture type classification.

Control circuit 80 may store each capture type classification, associated level of confidence and associated VCS pacing pulse amplitude (and/or pulse width) in memory 82 as capture threshold test results data. At block 612, control circuit 80 may be configured to determine a capture threshold for one or more capture types based on the stored capture threshold test results data. In various examples, when more than one capture type classification is stored in memory 82 for each VCS pacing pulse output, control circuit 80 may determine the capture type for the VCS pacing pulse output based on at least a threshold number or percentage of the capture type classifications being of one type and/or having at least a threshold confidence level.

For example, if therapy delivery circuit 84 delivers three VCS pacing pulses at each pacing pulse amplitude, control circuit 80 may require that at least two of the capture type classifications output by PCC 51 be the same classification in order to determine that capture type for the associated VCS pacing pulse amplitude. When the same capture type classification is made for at least 50%, 60%, 75%, 100% or other threshold percentage of the capture type classifications output by PCC 51 for a given pacing pulse output, control circuit 80 may determine that capture type for the given pacing pulse output. In some examples, control circuit 80 may additionally require that a threshold confidence level be met by the capture type classifications. For example, control circuit 80 may determine when at least 50% (or a higher percentage) of the capture type classifications output by PCC 51 for multiple VCS pacing pulses having the same pacing pulse output are the same capture type, and each of the matching capture types have a level of confidence of at least 80% or at least 90%. The matching capture type classifications meeting the threshold level of confidence may be stored in memory 82 as the capture type for the associated pacing pulse output. Other criteria or thresholds may be applied by control circuit 80 to the PCC 51 output for determining and storing the type of capture associated with each VCS pacing pulse output delivered during the capture threshold test.

Once the capture type is determined for each pacing pulse output by control circuit 80 based on the PCC 51 output, control circuit 80 may determine the capture threshold for each capture type at block 612. The capture threshold can be the lowest pacing pulse amplitude (when a fixed pulse width is used) for which a particular capture type is determined. In various examples, control circuit 80 may determine a NSHP capture threshold, a SHP capture threshold and/or VMO capture threshold depending on the types of capture determined for each pacing pulse output used during the capture threshold test as generally described above. As indicated previously herein, the types of capture detected during a capture threshold test may vary between patients and/or over time and not all capture types and associated capture thresholds may be determined.

At block 614, control circuit 80 may select an operating pacing pulse output based on the determined capture threshold(s). The selected operating pacing pulse output can be a safety margin above a capture threshold of a desired capture type. Control circuit 80 may adjust the pacing pulse amplitude and/or the pacing pulse width to the selected operating pacing pulse output at block 614. In some examples, control circuit 80 may set the operating pacing pulse amplitude to an amplitude that is a safety margin greater than a SHP capture threshold or a NSHP capture threshold. In some examples, when the SHP capture threshold is less than the NSHP capture threshold, control circuit 80 may set the operating pacing pulse amplitude to an amplitude that is greater than the SHP capture threshold but less than the NSHP capture threshold. In still other examples, control circuit 80 may set the operating pacing pulse amplitude to a safety margin greater than the NSHP capture threshold or greater than the SHP capture threshold, whichever is lower, to promote capture of at least a portion of the His-Purkinje system and to conserve IMD power source 98. In still other examples, control circuit 80 may set the operating pacing pulse output to be a safety margin greater than the NSHP capture threshold to minimize the likelihood of complete LOC of both the His-Purkinje system and the ventricular myocardium.

The operating pacing pulse output may be adjusted by control circuit 80 at block 614 and used by therapy delivery circuit 84 for delivering subsequent VCS pacing pulses according to a pacing therapy or pacing mode, at least until the next VCS capture check is performed or a programming change is made by a user. Control circuit 80 returns to block 601 to wait for the next time of a VCS capture check. The capture threshold test data, results, and or adjusted pacing pulse output may be stored in memory 82 and subsequently transmitted by IMD telemetry circuit 88, e.g., for receipt by external device 50, for display in a GUI and review by a clinician.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of circuits or components associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

The following examples are a non-limiting list of clauses in accordance with one or more techniques of this disclosure.

Example 1. A medical device system, comprising: a memory configured to store a first cardiac signal sensed following delivery of a ventricular conduction system (VCS) pacing pulse; processing circuitry configured to: receive the first cardiac signal sensed following delivery of the VCS pacing pulse; apply a pacing capture classification machine learning model to at least the first cardiac signal; determine, based on the applied pacing capture classification machine learning model, a capture type classification of the VCS pacing pulse from among a plurality of capture types; and generate an output based on the capture type classification; and a user interface configured to, in response to the generated output, present a representation of the capture type classification associated with the delivered ventricular conduction system pacing pulse.

Example 2. The medical device system of Example 1, wherein: the memory is further configured to store a first template beat signal corresponding to a first VCS pacing pulse output; and the processing circuitry is further configured to: input to the pacing capture classification machine learning model at least the first template beat signal and the first cardiac signal; and determine the capture type classification based on the pacing capture classification machine learning model applied to at least the first template beat signal and the first cardiac signal.

Example 3. The medical device system of any of Examples 1-2, wherein: the memory is further configured to store a first template beat signal corresponding to a first VCS pacing pulse output; and the processing circuitry is further configured to: determine a first template difference signal from the first cardiac signal and the first template beat signal; input at least the first template difference signal and the first cardiac signal to the pacing capture classification machine learning model; and determine the capture type classification based on the pacing capture classification machine applied to at least the first template difference signal and the first cardiac signal model.

Example 4. The medical device system of any of Examples 1-3, wherein: the memory is further configured to store a plurality of template beat signals, where each template beat signal corresponds to one VCS pacing pulse output of a plurality of VCS pacing pulse outputs; and the processing circuitry is further configured to: input each of the plurality of template beat signals and the first cardiac signal to the pacing capture classification machine learning model; and determine the capture type classification based on the pacing capture classification machine learning model applied to at least the plurality of template beat signals and the first cardiac signal.

Example 5. The medical device system of any of Examples 1-4, wherein: the memory is further configured to store a plurality of template beat signals, where each template beat signal corresponds to one VCS pacing pulse output of a plurality of VCS pacing pulse outputs; and the processing circuitry is further configured to: determine a plurality of template difference signals by determining a template difference signal from each one of the plurality of template beat signals and the first cardiac signal; and input the plurality of template difference signals and the first cardiac signal to the pacing capture classification machine learning model; and determine the capture type classification based on the pacing capture classification machine learning model applied to at least the plurality of template difference signals and the first cardiac signal.

Example 6. The medical device of any of Examples 4-5, wherein the memory is further configured to store the plurality of template beat signals by storing a first template beat signal corresponding to a first VCS pacing pulse output, a second template beat signal corresponding to a second VCS pacing pulse output that is less than the first VCS pacing pulse output, and a third template beat signal corresponding to a third VCS pacing pulse output that is intermediate the first and second VCS pacing pulse outputs.

Example 7. The medical device system of any of Examples 1-6, wherein the processing circuitry is further configured to: determine a derivative signal from the first cardiac signal; input the first cardiac signal to the pacing capture classification machine learning model by inputting at least the derivative signal; and determine the capture type classification based on the pacing capture classification machine learning model applied to at least the derivative signal.

Example 8. The medical device system of any of Examples 1-7, wherein: the memory is further configured to store a second cardiac signal sensed following delivery of the VCS pacing pulse, wherein the first cardiac signal being sensed by a first sensing electrode vector and the second cardiac signal being sensing by a second sensing electrode vector different than the first sensing electrode vector; and the processing circuitry is further configured to: input each of the first cardiac signal and the second cardiac signal to the pacing capture classification machine learning model; and determine the capture type classification based on the pacing capture classification machine learning model applied to at least the first cardiac signal and the second cardiac signal.

Example 9. The medical device system of any of Examples 1-8, wherein: the memory is further configured to store a pacing pulse output of the delivered VCS pacing pulse; and the processing circuitry is further configured to: input at least the pacing pulse output and the first cardiac signal to the pacing capture classification machine learning model; and determine the capture type classification based on the pacing capture classification machine learning model applied to at least the pacing pulse output and the first cardiac signal.

Example 10. The medical device system of any of Examples 1-9, wherein the processing circuitry is further configured to: determine a feature of the first cardiac signal; input the feature of the first cardiac signal and the first cardiac signal to the pacing capture classification machine learning model; and determine the capture type classification based on the pacing capture classification machine learning model applied to at least the feature of the first cardiac signal and the first cardiac signal.

Example 11. The medical device system of any of Examples 1-10, wherein the processing circuitry is further configured to: receive training cardiac signal datasets obtained from a plurality of patients, the training cardiac signal datasets comprising a plurality of training cardiac signals each sensed following delivery of a VCS pacing pulse, wherein the VCS pacing pulses associated with the plurality of training cardiac signals comprise VCS pacing pulses delivered at a plurality of different pacing pulse outputs; train the pacing capture classification machine learning model with the training cardiac signal datasets according to a machine learning algorithm; and apply the pacing capture classification machine learning model trained with the training cardiac signal datasets to at least the first cardiac signal.

Example 12. The medical device system of any of Examples 1-11, wherein the processing circuitry is further configured to: receive a plurality of cardiac signals comprising the first cardiac signal, each of the plurality of cardiac signals associated with a VCS pacing pulse, wherein the VCS pacing pulses associated with the plurality of cardiac signals comprise VCS pacing pulses delivered at a plurality of different pacing pulse outputs; input each of the plurality of cardiac signals to the pacing capture classification machine learning model; determine a capture type classification of each of the VCS pacing pulses delivered at the plurality of different pacing pulse outputs associated with the plurality of cardiac signals based on the pacing capture classification machine learning model; and determine a capture threshold for at least one capture type of the plurality of capture types based on the capture type classifications.

Example 13. The medical device system of Example 12, wherein the user interface is further configured to present the capture threshold for the at least one capture type.

Example 14. The medical device system of any of Examples 12-13, wherein the processing circuitry is further configured to determine an operating pacing pulse output based on the capture threshold determined for the at least one capture type.

Example 15. The medical device system of Example 14, further comprising a therapy delivery circuit configured to generate VCS pacing pulses according to the operating pacing pulse output.

Example 16. The medical device system of any of Examples 1-15, wherein the processing circuitry is further configured to determine the capture type classification as one of selective His-Purkinje system capture without ventricular myocardial capture, non-selective His-Purkinje system capture with ventricular myocardial capture, ventricular myocardial capture without His-Purkinje system capture, or loss of capture.

Example 17. A method, comprising: storing in a memory a first cardiac signal sensed following delivery of a ventricular conduction system (VCS) pacing pulse; receiving by processing circuitry the first cardiac signal sensed following delivery of the VCS pacing pulse; applying a pacing capture classification machine learning model to at least the first cardiac signal; determining, based on the applied pacing capture classification machine learning model, a capture type classification of the VCS pacing pulse from among a plurality of capture types; generating by the processing circuitry an output based on the capture type classification; and presenting by a user interface a representation of the capture type classification associated with the delivered VCS pacing pulse in response to the generated output.

Example 18. The method of Example 17, further comprising: storing in the memory a first template beat signal corresponding to a first VCS pacing pulse output; inputting to the pacing capture classification machine learning model at least the first template beat signal and the first cardiac signal; and determining the capture type classification based on the pacing capture classification machine learning model applied to at least the first template beat signal and the first cardiac signal.

Example 19. The method of any of Examples 17-18, further comprising: storing in the memory a first template beat signal corresponding to a first VCS pacing pulse output; determining by the processing circuitry a first template difference signal from the first cardiac signal and the first template beat signal; inputting at least the first template difference signal and the first cardiac signal to the pacing capture classification machine learning model; and determining the capture type classification based on the pacing capture classification machine learning model applied to at least the first template difference signal and the first cardiac signal.

Example 20. The method of any of Examples 17-19, further comprising: storing in the memory a plurality of template beat signals, where each template beat signal corresponds to one VCS pacing pulse output of a plurality of VCS pacing pulse outputs; inputting each of the plurality of template beat signals and the first cardiac signal to the pacing capture classification machine learning model; and determining the capture type classification based on the pacing capture classification machine learning model applied to at least the plurality of template beat signals and the first cardiac signal.

Example 21. The method of any of Examples 17-20, further comprising: storing in the memory a plurality of template beat signals, where each template beat signal corresponds to one VCS pacing pulse output of a plurality of VCS pacing pulse outputs; determining a plurality of template difference signals by determining a template difference signal from each one of the plurality of template beat signals and the first cardiac signal; inputting the plurality of template difference signals and the first cardiac signal to the pacing capture classification machine learning model; and determining the capture type classification based on the pacing capture classification machine learning model applied to at least the plurality of template difference signals and the first cardiac signal.

Example 22. The method of any of Examples 20-21, further comprising storing the plurality of template beat signals by storing a first template beat signal corresponding to a first VCS pacing pulse output, a second template beat signal corresponding to a second VCS pacing pulse output that is less than the first VCS pacing pulse output, and a third template beat signal corresponding to a third VCS pacing pulse output that is intermediate the first and second VCS pacing pulse outputs.

Example 23. The method of any of Examples 17-22, further comprising: determining a derivative signal from the first cardiac signal; inputting the first cardiac signal to the pacing capture classification machine learning model by inputting at least the derivative signal; and determining the capture type classification based on the pacing capture classification machine learning model applied to at least the derivative signal.

Example 24. The method of any of Examples 17-23, further comprising: storing a second cardiac signal sensed following delivery of the ventricular conduction system (VCS) pacing pulse, wherein the first cardiac signal being sensed by a first sensing electrode vector and the second cardiac signal being sensing by a second sensing electrode vector different than the first sensing electrode vector inputting each of the first cardiac signal and the second cardiac signal to the pacing capture classification machine learning model; and determining the capture type classification based on the pacing capture classification machine learning model applied to at least the first cardiac signal and the second cardiac signal.

Example 25. The method of any of Examples 17-24, further comprising: storing in the memory a pacing pulse output of the delivered VCS pacing pulse; inputting at least the pacing pulse output and the first cardiac signal to the pacing capture classification machine learning model; and determining the capture type classification based on the pacing capture classification machine learning model applied to at least the pacing pulse output and the first cardiac signal.

Example 26. The method of any of Examples 17-25, further comprising: determining a feature of the first cardiac signal; inputting the feature of the first cardiac signal and the first cardiac signal to the pacing capture classification machine learning model; and determining the capture type classification based on the pacing capture classification machine learning model applied to at least the feature of the first cardiac signal and the first cardiac signal.

Example 27. The method of any of Examples 17-26, further comprising: receiving by the processing circuitry training cardiac signal datasets obtained from a plurality of patients, the training cardiac signal datasets comprising a plurality of training cardiac signals each sensed following delivery of a VCS pacing pulse, wherein the VCS pacing pulses associated with the plurality of training cardiac signals comprise VCS pacing pulses delivered at a plurality of different pacing pulse outputs; training the pacing capture classification machine learning model with the training cardiac signal datasets according to a machine learning algorithm; and applying the pacing capture classification machine learning model trained with the training cardiac signal datasets to at least the first cardiac signal.

Example 28. The method of any of Examples 17-27, further comprising: receiving by the processing circuitry a plurality of cardiac signals comprising the first cardiac signal, each of the plurality of cardiac signals associated with a VCS pacing pulse, wherein the VCS pacing pulses associated with the plurality of cardiac signals comprise VCS pacing pulses delivered at a plurality of different pacing pulse outputs; inputting each of the plurality of cardiac signals to the pacing capture classification machine learning model; determining a capture type classification of each of the VCS pacing pulses delivered at the plurality of different pacing pulse outputs associated with the plurality of cardiac signals based on the pacing capture classification machine learning model; and determining a capture threshold for at least one capture type of the plurality of capture types based on the capture type classifications.

Example 29. The method of Example 28, further comprising presenting by the user interface the capture threshold for the at least one capture type.

Example 30. The method of any of Examples 28-29, further comprising determining an operating pacing pulse output based on the capture threshold determined for the at least one capture type.

Example 31. The method of Example 30, further comprising generating VCS pacing pulses according to the operating pacing pulse output.

Example 32. The method of any of Examples 17-31, further comprising determining the capture type classification as one of selective His-Purkinje system capture without ventricular myocardial capture, non-selective His-Purkinje system capture with ventricular myocardial capture, ventricular myocardial capture without His-Purkinje system capture, or loss of capture.

Example 33. A non-transitory, computer-readable storage medium storing a set of instructions which, when executed by processing circuitry of a medical device system, cause the medical device system to: store in a memory of the medical device system a cardiac signal sensed following delivery of a ventricular conduction system (VCS) pacing pulse; apply a pacing capture classification machine learning model to at least the cardiac signal; determine, based on the applied pacing capture classification machine learning model, a capture type classification of the VCS pacing pulse from among a plurality of capture types; generate an output based on the capture type classification; and present a representation of the determined capture type associated with the delivered ventricular conduction system pacing pulse by a user interface of the medical device system.

Example 34. A medical device system, comprising: a sensing circuit configured to sense a cardiac electrical signal; a therapy delivery circuit configured to deliver a ventricular conduction system (VCS) pacing pulse; processing circuitry configured to: receive the cardiac electrical signal; apply a machine learning model to at least the sensed cardiac electrical signal, the machine learning model being trained using a plurality of cardiac signal datasets to determine a pacing capture type; determine, based on the applied machine learning model, a pacing capture type of the VCS pacing pulse; and a user interface configured to present a representation of the determined pacing capture type of the VCS pacing pulse.

Example 35. A medical device system, comprising: a sensing circuit configured to sense at least one cardiac electrical signal; a therapy delivery circuit configured to deliver ventricular conduction system (VCS) pacing pulses; a memory configured to store a cardiac signal segment sensed by the sensing circuit during at least a post-pace time interval following delivery of a first VCS pacing pulse delivered by the therapy delivery circuit; and processing circuitry configured to: apply a machine learning model to at least the first cardiac signal segment; determine, based on the machine learning model, a pacing capture type of the first VCS pacing pulse from among a plurality of capture types; select an operating pacing pulse output based on at least the determined pacing capture type; wherein the therapy delivery circuit is further configured to deliver a second VCS pacing pulse according to the selected operating pacing pulse output.

1. A medical device system, comprising:
a memory configured to store a first cardiac signal sensed following delivery of a ventricular conduction system (VCS) pacing pulse;
processing circuitry configured to:
receive the first cardiac signal sensed following delivery of the VCS pacing pulse;
apply a pacing capture classification machine learning model to at least the first cardiac signal;
determine, based on the applied pacing capture classification machine learning model, a capture type classification of the VCS pacing pulse from among a plurality of capture types; and
generate an output based on the capture type classification; and
a user interface configured to, in response to the generated output, present a representation of the capture type classification associated with the delivered ventricular conduction system pacing pulse.

2. The medical device system of claim 1, wherein:
the memory is further configured to store a first template beat signal corresponding to a first VCS pacing pulse output; and
the processing circuitry is further configured to:
input to the pacing capture classification machine learning model at least the first template beat signal and the first cardiac signal; and
determine the capture type classification based on the pacing capture classification machine learning model applied to at least the first template beat signal and the first cardiac signal.

3. The medical device system of claim 1, wherein:
the memory is further configured to store a first template beat signal corresponding to a first VCS pacing pulse output; and
the processing circuitry is further configured to:
determine a first template difference signal from the first cardiac signal and the first template beat signal;
input at least the first template difference signal and the first cardiac signal to the pacing capture classification machine learning model; and
determine the capture type classification based on the pacing capture classification machine applied to at least the first template difference signal and the first cardiac signal.

4. The medical device system of claim 2, wherein:
the memory is further configured to store a plurality of template beat signals comprising the first template beat signal, where each of the plurality of template beat signal corresponds to one VCS pacing pulse output of a plurality of VCS pacing pulse outputs; and
the processing circuitry is further configured to:
input each of the plurality of template beat signals and the first cardiac signal to the pacing capture classification machine learning model; and
determine the capture type classification based on the pacing capture classification machine learning model applied to at least the plurality of template beat signals and the first cardiac signal.

5. The medical device system of claim 3, wherein:
the memory is further configured to store a plurality of template beat signals comprising the first template beat signal, where each template beat signal corresponds to one VCS pacing pulse output of a plurality of VCS pacing pulse outputs; and
the processing circuitry is further configured to:
determine a plurality of template difference signals, comprising the first template difference signal, by determining a template difference signal from each one of the plurality of template beat signals and the first cardiac signal; and
input the plurality of template difference signals and the first cardiac signal to the pacing capture classification machine learning model; and
determine the capture type classification based on the pacing capture classification machine learning model applied to at least the plurality of template difference signals and the first cardiac signal.

6. (canceled)

7. The medical device system of claim 1 wherein the processing circuitry is further configured to:
determine a derivative signal from the first cardiac signal;
input the first cardiac signal to the pacing capture classification machine learning model by inputting at least the derivative signal; and
determine the capture type classification based on the pacing capture classification machine learning model applied to at least the derivative signal.

8. (canceled)

9. The medical device system of claim 1 wherein:
the memory is further configured to store a pacing pulse output of the delivered VCS pacing pulse; and
the processing circuitry is further configured to:
input at least the pacing pulse output and the first cardiac signal to the pacing capture classification machine learning model; and
determine the capture type classification based on the pacing capture classification machine learning model applied to at least the pacing pulse output and the first cardiac signal.

10. The medical device system of claim 1 wherein the processing circuitry is further configured to:
determine a feature of the first cardiac signal;
input the feature of the first cardiac signal and the first cardiac signal to the pacing capture classification machine learning model; and
determine the capture type classification based on the pacing capture classification machine learning model applied to at least the feature of the first cardiac signal and the first cardiac signal.

11. The medical device system of claim 1 wherein the processing circuitry is further configured to:
receive training cardiac signal datasets obtained from a plurality of patients, the training cardiac signal datasets comprising a plurality of training cardiac signals each sensed following delivery of a VCS pacing pulse, wherein the VCS pacing pulses associated with the plurality of training cardiac signals comprise VCS pacing pulses delivered at a plurality of different pacing pulse outputs;
train the pacing capture classification machine learning model with the training cardiac signal datasets according to a machine learning algorithm; and
apply the pacing capture classification machine learning model trained with the training cardiac signal datasets to at least the first cardiac signal.

12. The medical device system of claim 1 wherein:
the processing circuitry is further configured to:
receive a plurality of cardiac signals comprising the first cardiac signal, each of the plurality of cardiac signals associated with a VCS pacing pulse, wherein the VCS pacing pulses associated with the plurality of cardiac signals comprise VCS pacing pulses delivered at a plurality of different pacing pulse outputs;
input each of the plurality of cardiac signals to the pacing capture classification machine learning model;
determine a capture type classification of each of the VCS pacing pulses delivered at the plurality of different pacing pulse outputs associated with the plurality of cardiac signals based on the pacing capture classification machine learning model;
determine a capture threshold for at least one capture type of the plurality of capture types based on the capture type classifications; and
determine an operating pacing pulse output based on the capture threshold determined for the at least one capture type; and
the user interface is further configured to generate a display of the operating pacing pulse output for a user to accept and confirm.

13. (canceled)

14. (canceled)

15. A non-transitory, computer-readable storage medium storing a set of instructions which, when executed by processing circuitry of a medical device system, cause the medical device system to:
store in a memory of the medical device system a first cardiac signal sensed following delivery of a ventricular conduction system (VCS) pacing pulse;
apply a pacing capture classification machine learning model to at least the first cardiac signal;
determine, based on the applied pacing capture classification machine learning model, a capture type classification of the VCS pacing pulse from among a plurality of capture types;
generate an output based on the capture type classification; and
in response to the generated output, present a representation of the determined capture type associated with the delivered ventricular conduction system pacing pulse by a user interface of the medical device system.

16. The storage medium of claim 15 further comprising instructions that cause the medical device system to:
store a first template beat signal corresponding to a first VCS pacing pulse output;
input to the pacing capture classification machine learning model at least the first template beat signal and the first cardiac signal; and
determine the capture type classification based on the pacing capture classification machine learning model applied to at least the first template beat signal and the first cardiac signal.

17. The storage medium of claim 16 further comprising instructions that cause the medical device system to:
store a plurality of template beat signals comprising the first template beat signal, where each of the plurality of template beat signal corresponds to one VCS pacing pulse output of a plurality of VCS pacing pulse outputs;
input each of the plurality of template beat signals and the first cardiac signal to the pacing capture classification machine learning model; and
determine the capture type classification based on the pacing capture classification machine learning model applied to at least the plurality of template beat signals and the first cardiac signal.

18. The storage medium of claim 15 further comprising instructions that cause the medical device system to:
store a first template beat signal corresponding to a first VCS pacing pulse output;
determine a first template difference signal from the first cardiac signal and the first template beat signal;
input at least the first template difference signal and the first cardiac signal to the pacing capture classification machine learning model; and
determine the capture type classification based on the pacing capture classification machine applied to at least the first template difference signal and the first cardiac signal.

19. The storage medium of claim 18 further comprising instructions that cause the medical device system to:
store a plurality of template beat signals comprising the first template beat signal, where each template beat signal corresponds to one VCS pacing pulse output of a plurality of VCS pacing pulse outputs;
determine a plurality of template difference signals, comprising the first template difference signal, by determining a template difference signal from each one of the plurality of template beat signals and the first cardiac signal;
input the plurality of template difference signals and the first cardiac signal to the pacing capture classification machine learning model; and
determine the capture type classification based on the pacing capture classification machine learning model applied to at least the plurality of template difference signals and the first cardiac signal.

20. The storage medium of claim 15 further comprising instructions that cause the medical device system to:
determine a derivative signal from the first cardiac signal;
input the first cardiac signal to the pacing capture classification machine learning model by inputting at least the derivative signal; and
determine the capture type classification based on the pacing capture classification machine learning model applied to at least the derivative signal.

21. The storage medium of claim 15 further comprising instructions that cause the medical device system to:
store a pacing pulse output of the delivered VCS pacing pulse;
input at least the pacing pulse output and the first cardiac signal to the pacing capture classification machine learning model; and
determine the capture type classification based on the pacing capture classification machine learning model applied to at least the pacing pulse output and the first cardiac signal.

22. The storage medium of claim 15 further comprising instructions that cause the medical device system to:
determine a feature of the first cardiac signal;
input the feature of the first cardiac signal and the first cardiac signal to the pacing capture classification machine learning model; and
determine the capture type classification based on the pacing capture classification machine learning model applied to at least the feature of the first cardiac signal and the first cardiac signal.

23. The storage medium of claim 15 further comprising instructions that cause the medical device system to:
receive training cardiac signal datasets obtained from a plurality of patients, the training cardiac signal datasets comprising a plurality of training cardiac signals each sensed following delivery of a VCS pacing pulse, wherein the VCS pacing pulses associated with the plurality of training cardiac signals comprise VCS pacing pulses delivered at a plurality of different pacing pulse outputs;
train the pacing capture classification machine learning model with the training cardiac signal datasets according to a machine learning algorithm; and
apply the pacing capture classification machine learning model trained with the training cardiac signal datasets to at least the first cardiac signal.

24. The storage medium of claim 15 further comprising instructions that cause the medical device system to:
receive a plurality of cardiac signals comprising the first cardiac signal, each of the plurality of cardiac signals associated with a VCS pacing pulse, wherein the VCS pacing pulses associated with the plurality of cardiac signals comprise VCS pacing pulses delivered at a plurality of different pacing pulse outputs;
input each of the plurality of cardiac signals to the pacing capture classification machine learning model;
determine a capture type classification of each of the VCS pacing pulses delivered at the plurality of different pacing pulse outputs associated with the plurality of cardiac signals based on the pacing capture classification machine learning model;
determine a capture threshold for at least one capture type of the plurality of capture types based on the capture type classifications; and
determine an operating pacing pulse output based on the capture threshold determined for the at least one capture type; and
generate a display of the operating pacing pulse output for a user to accept and confirm.
